SEQUENCE LISTING (2 sequences)

SEQ ID NO: 1 - protein, 12 aa, synthetic construct
HLLQPTQNPFRN

SEQ ID NO: 2 - protein, 5 aa, synthetic construct
GGGGS

(19) United States
(12) Patent Application Publication
(10) Pub. No.: US 2026/0251646 A1
(43) Pub. Date: Aug. 27, 2026

(54) BIOSENSOR TESTING SYSTEM AND METHODS OF USE

(71) Applicants: Quidel Corporation, San Diego, CA (US); University of Dayton, Dayton, OH (US)

(72) Inventors: Richard L. Egan, Sparks, NV (US); Christopher Muratore, Kettering, OH (US)

(21) Appl. No.: 18/861,517

(22) PCT Filed: Apr. 28, 2023
(86) PCT No.: PCT/US2023/066404

§ 371 Date: Oct. 29, 2024

Related U.S. Application Data
(60) Provisional application No. 63/336,809, filed on Apr. 29, 2022.

(51) Int. Cl. G01N 33/543; G01N 27/12

(52) U.S. Cl. CPC G01N 33/5438; G01N 27/125

(57) ABSTRACT

A system comprised of a sensor chip and a reader is provided. The sensor chip has a sensing region with a detection agent that binds or associates with an analyte of interest in a sample. If present in the sample, interaction between the analyte of interest and the detection agent causes a change in resistance or conductivity of the sensing region, where the change or lack of change indicates presence or absence of the analyte of interest.

### CROSS-REFERENCE TO RELATED APPLICATIONS

This application claims the benefit of U.S. Provisional Application No. 63/336,809, filed Apr. 29, 2022, incorporated herein by reference in its entirety.

### REFERENCE TO SEQUENCE LISTING XML

This Application incorporates by reference the Sequence Listing XML file submitted via the patent office electronic filing system having the file name “041896-1343-8168W000_SEQ XML” and created on 2023 Apr. 27 with a file size of 2,580 bytes.

### TECHNICAL FIELD

The subject matter described herein relates to a sensitive and rapid diagnostic assay for detection of one or more infectious agents and methods of making and using the same.

### BACKGROUND

Current sensors that are used to sense/detect materials such as viruses, bacteria and chemicals are costly to make and/or use. Thus, the use of such sensors is more limited than desired. The problems associated with the current sensors were the processing challenges associated with achieving the required size of the active region of the sensor, sensor chemical and structural uniformity from sensor to sensor that were driven by the complexity of the chemical and physical steps of current production processes.

Ultrathin materials can be crystalline and may consist of multiple layers or of a single layer of atoms, sometimes 3-8 molecules in a layer, and can be referred to as two-dimensional (2D) or single layer materials. Applications for 2D materials and their heterostructures in fields such as communications, high speed computing, sensing, and energy harvesting are currently limited by the absence of direct and repeatable synthesis methods for cost effective device fabrication. While conducting (e.g., graphene, tantalim sulfide (TaS₂)), and semiconducting (e.g., molybdenum disulfide (MoS₂), tungsten disulfide (WS₂)) 2D materials are being rapidly advanced for next generation 2D devices, ultrathin and high strength dielectric materials for transistor gates, capacitors, memory devices, and barrier layers for electrical and ambient environment isolation are far less developed.

This circumstance is primarily a result of the fundamental challenge in synthesis of ultrathin insulating materials at moderate temperatures (generally less than 900° C.) needed for direct growth over large lateral dimensions. In silicon-based electronics, silicon dioxide (SiO₂), prepared by plasma enhanced chemical vapor deposition, has proven to be a suitable dielectric material due to the large band gap (9 eV), well-matched interfacial properties with silicon, and simple, repeatable processing. However, there is a continuing desire to develop additional ultrathin dielectric materials which unique 2D benefits such as optical transparency and mechanical flexibility, for use in flexible electronic devices and other premium areas of nanotechnology innovation. Attempts to provide suitable ultrathin dielectric alternatives have included atomic layer deposition of HfO₂ (hafnium (IV) oxide) and Al₂O₃ (aluminum oxide) and thermal activated growth of crystalline hexagonal form boron nitride (h-BN) by chemical vapor deposition. To date, however, such ultrathin dielectrics have been found to suffer from significant scaling, process tuning, and pinhole-free uniformity challenges.

During the COVID pandemic, diagnostic tests were developed to quickly detect fragments of proteins found on or within the SAR CoV-2 virus by testing samples collected from the nasal cavity using swabs. However, there were reports of patients waiting in line for test results or waiting for days to receive accurate test results. This has spotlighted the need for a generational leap in assays capable of rapid, simple detection of analytes of interest, such as infectious agents, and optionally processes for reporting test results from the test assay, while assuring performance and accuracy.

Accordingly, there is a need for a next generation diagnostic test, that can be used at home or in clinical settings, such as point-of-care (POC) or laboratories, for detecting presence or absence of one or more analytes of interest, such as infectious agents, in a sample from a subject, and that has greatly improved performance (sensitivity and specificity) and dramatically shortened total assay times.

### BRIEF SUMMARY

The following aspects and embodiments thereof described and illustrated below are meant to be exemplary and illustrative, not limiting in scope.

In one aspect, a system comprising a cassette and a reader is provided. The cassette comprises a sensor chip, the sensor chip comprised of a substrate with a first planar surface and a second planar surface, electrical contacts, a sensing region comprised of semiconducting transition metal, and a detection agent that interacts with an analyte of interest. The reader comprises a port configured to receive the cassette, the reader comprising a source of electrical current, a detector and a transmitter.

In one embodiment, the detector is capable of converting resistance of the sensing region to electronic signal.

In one embodiment, the detector is capable of detecting resistance of a sensing region on the sensor chip.

In one embodiment, the transmitter is capable of transmitting an electronic signal. In one embodiment, the transmitter is a transceiver. In one embodiment, the transmitter or transceiver is wireless. In one embodiment, the transmitter or transceiver comprises a wired or physical connection.

In one embodiment, the source of electrical current is capable of applying direct current or alternating current. In one embodiment, the source of electrical current is capable of applying from about 1 pA to 100 nA to the electrical contacts.

In one embodiment, the reader is configured for insertion into a universal serial bus (USB). In one embodiment, the reader is configured to power on in response to insertion of a cassette into the port. In one embodiment, the reader comprises a memory and a processor.

In one embodiment, the reader comprises an algorithm stored in memory, the algorithm is initiated in response to (i) insertion of a cassette into the port or (ii) placement of a sample onto the cassette.

In one embodiment, the algorithm instructs the transmitter to transmit a signal to an electronic device that is separate from the system.

In one embodiment, the electronic device is a user mobile device.

In one embodiment, the system further comprises a software application downloadable to an electronic device separate from the system, the software application configured to respond to signal transmitted from the reader.

In one embodiment, the electronic device separate from the system comprises a memory and a processor, wherein the software application is retained in the memory and comprises an executable file comprising an algorithm with instructions for interaction between the reader and the external device separate from the system.

In one embodiment, the substrate is a flexible glass.

In one embodiment, the electrical contacts are comprised of a conductive metal. In one embodiment, the metal is selected from the group consisting of Cr, Ti, Sn Ni, V, Hf, W, Mo, MoS₂, Nb, Au, Ag, Cu and Pt, and combinations thereof.

In one embodiment, the transition metal is selected from the group consisting of molybdenum, tungsten, niobium, tantalum, vanadium, titanium, chromium, iron, rhodium, hafnium, rhenium and mixtures thereof.

In one embodiment, the transition metal further comprises an element selected from the group consisting of nitrogen, sulfur, selenium, phosphorous, hydrogen and carbon.

In one embodiment, the transition metal is molybdenum.

In one embodiment, the sensing region is comprised of molybdenum disulfide (MoS₂).

In one embodiment, the molybdenum disulfide is crystalline.

In one embodiment, detection agent has specific binding for the analyte of interest. In one embodiment, the detection agent is an antibody fragment. In one embodiment, the detection agent is a fusion protein of a linker and an antibody fragment. In one embodiment, the detection agent is a fusion protein of two linkers and an antibody fragment.

In one embodiment, the antibody fragment comprises a first heavy chain and a second heavy chain, wherein one of said two linkers is attached to a terminal end of the first heavy chain and one of the two linkers is attached to a terminal end of the second heavy chain. In one embodiment the antibody fragment comprises a constant heavy chain and a constant light chain, and one of said two linkers is attached to the constant heavy chain and one of the two linkers is attached to the constant light chain.

In one embodiment, the cassette and/or the sensor chip comprises an identifier that encodes information regarding the sensor chip. In one embodiment, the information identifies an analyte of interest with which the detection agent interacts.

In one embodiment, the cassette comprises contacts for operable connection with the source of electrical current in the reader.

In one embodiment, the cassette comprises a plurality of sensor chips.

In one embodiment, the sensor chip comprises a plurality of sensing regions.

In one embodiment, the sensor chip comprises a plurality of sensing regions, wherein each sensing region comprises an immobilized detection agent.

In one embodiment, the detection agent in each sensing region of the plurality of sensing regions interacts with the same analyte of interest.

In one embodiment, the detection agent in each sensing region of the plurality of sensing regions interacts with a different analyte of interest.

In one embodiment, the plurality of sensing regions comprises between 3-25 sensing regions.

In one embodiment, each sensing region in the plurality of sensing regions has a dedicated pair of electrical contacts.

In one embodiment, the sensor chip comprises a plurality of sensing regions, wherein at least one sensing region comprises a detection agent that serves as an assay control.

In one embodiment, the cassette comprises contacts for operable connection with the source of electrical current in the reader.

In one embodiment, the reader further comprises one or more indicators.

In one embodiment, the one or more indicators is a light, an audible signal and/or a sensory signal.

In one embodiment, the cassette comprising a component to provide heat to the sensor chip.

In an aspect, a method for detecting an analyte of interest is provided. The method comprises providing a cassette comprising a sensor chip comprised of a substrate with a first planar surface and a second planar surface, electrical contacts, a sensing region comprised of a semiconducting transition metal, and a detection agent that interacts with an analyte of interest; evaluating a first resistance of the sensing region; contacting the sensing region with a sample suspected of comprising an analyte of interest that specifically binds the detection agent; evaluating a second resistance of the sensing region; and detecting binding of the analyte of interest to the detection agent if the second resistance is different from the first resistance.

In an aspect, a method for detecting an analyte of interest is provided. The method comprises providing a cassette comprising a sensor chip comprised of a substrate with a first planar surface and a second planar surface, electrical contacts, a sensing region comprised of a semiconducting transition metal, and a detection agent that interacts with an analyte of interest; inserting the cassette in a reader having a port configured to receive the cassette, the reader comprising a source of electricity, a detector, and a transmitter; measuring with the detector a first resistance of the sensing region by causing the source of electricity to apply electrical current to the electrical contacts; depositing a sample, or instructing to deposit a sample, on the cassette to cause sample to contact the sensing region; measuring with the detector a second resistance of the sensing region by causing the source of electricity to apply electrical current to the electrical contacts; and detecting binding of the analyte of interest to the detection agent if the second resistance is different from the first resistance.

In one embodiment, the methods further comprise transmitting with the transmitter data associated with the first resistance, the second resistance, or both to an electronic device.

In one embodiment, the electronic device comprises a memory and a processor, and retained in the memory is a software application comprising an algorithm for analyzing the data.

In one embodiment, binding of the analyte of interest to the detection agent is detected if the second resistance is lower than the first resistance.

In one embodiment, binding of the analyte of interest to the detection agent is detected if the second resistance is higher than the first resistance.

In one embodiment, depositing a sample, or instructing to deposit a sample, comprises depositing a sample selected from the group of blood, urine, sweat, mucus, nasal discharge, saliva, feces, soil, waste stream, and food.

In one embodiment, depositing a sample, or instructing to deposit a sample, comprises depositing a sample selected from the group of blood, urine, sweat, mucus, nasal discharge, saliva, and feces that has been processed prior to said depositing.

In an aspect, a testing system for detection of one or more analytes of interest is provided. This system comprises a cassette which comprises sensor chip and a reader. In embodiments, the reader comprises a transmitter or transceiver to communicate with a receiving device that is external from the system. The sensor chip and/or the cassette can comprise a symbol or identifier, such as a universal product code, an electronic product code, a serial number, a bar code, a two-dimensional quick response (QR) code. In embodiments, the reader can comprise a camera to image or “read” the symbol or the identifier on each test cassette and transmit image to a receiving device. When the test cassette is inserted into the reader, the reader scans the symbol or identifier and transmits a test result, obtained from, on or by the test cassette, to a receiving member or means.

In some embodiments, the analyte of interest is an infectious agent. In an embodiment, the infectious agent is a virus, a bacteria, or a fungus. In other embodiments, the analyte of interest is a drug, such as a therapeutic drug or a drug of abuse. In embodiments, the analyte of interest is an antibody, a protein, or a peptide. In embodiments, the analyte of interest is a nucleotide or sequence of nucleotides. In embodiments, the analyte of interest is a nucleic acid strand or strands. In embodiments, the analyte of interest is a strand of DNA or RNA.

In other embodiment, the sensor chip in the test cassette is configured for detection of one or more analytes of interest, such as one or more analytes of interested selected from a nucleic acid, an antibody, a protein, a peptide, a therapeutic agent, or a drug of abuse. In embodiments, the nucleic acid is a DNA or an RNA sequence.

In some embodiments, the sensor chip is a 2-dimentional (2D) MoS₂ sensor chip. In certain embodiments, the 2D MoS₂ sensor chip may be functionalized by depositing onto the sensor chip a reagent, one or more assay specific reagents, and/or a mixture of assay specific reagents. In embodiments, the reagent, the assay specific reagents, and/or the mixture of assay specific reagents is deposited on the sensor chip via depositing discrete drops of the reagent, also referred to as spotting or drop coating. In embodiments, the discrete drops of reagents are in the pico-liter to nanoliter range, where more than one drop can be deposited in a specific position on the sensor surface. In other embodiments, before or after depositing with a reagent and/or a mixture of assay specific reagents, the sensor chip is treated to anneal and/or crystalize the MoS₂. In an embodiment, the sensor chip is treated by heating to a temperature of between about 275-550° C. or between about 300-500° C. or by exposing to a laser.

In some embodiments, the reagent, or the mixture of assay specific reagents, comprises a monoclonal antibody (mAb) expressed recombinantly in the form of recombinantly expressed antigen binding fragment (FAB) (rFABs). In embodiments, the rFAB is a F(ab′)₂ fragment, an Fab′ fragment, a Fab fragment, or an Fv fragment. In certain embodiments, the rFAB may comprise a linker peptide sequence, such as the linker peptide sequence HLLQPTQNPFRN (SEQ ID NO: 1). In some embodiments, each rFAB may be specific for an analyte of interest, such as an infectious agent, and the sensor chip may comprise multiple sensors that are deposited or spotted on the sensor chip surface with different rFABs specific for detection, if present, of different analytes of interest. In embodiments, one or more rFABs deposited or spotted on the sensor chip surface is a recombinantly expressed fusion protein of a linker peptide, such as SEQ ID NO: 1 or a linker peptide with 90%, 80% or 70% sequence identity to SEQ ID NO: 1.

In some embodiments, the reader may be a handheld reader that is operated by battery and capable of wireless communications, such as a wireless internet connection (Wifi), Bluetooth®, ZigBee or Z-Wave®.

In some embodiments, the receiving device, typically a user's electronic device, may be any mobile or non-mobile electronic device, such as a smartphone, a smartwatch, a tablet, a notebook, a laptop computer, or any other mobile device, or a computing device, such as desktop computer. The receiving device includes a processor, a memory, a transceiver, a display and a camera. The transceiver can be any type of wired or wireless hardware, firmware or software for enabling communication with the reader and other devices, such as via a wireless internet connection (Wifi), Bluetooth®, ZigBee or Z-Wave® or a wired connection, such an ethernet or USB. The receiving device can store in its memory for execution by its processor a user application designed and configured to interact with the reader, for example to prompt and/or guide a user to take certain actions on the test cassette or reader in order to obtain a test result being transmitted from the reader to the receiving device.

In some embodiments, the test results may include measurements of the changes in the electrical conductivity of the sensors during selective binding of the one or more analytes of interest to an rFAB.

In still another aspect, a method of producing a sensor chip for the testing system is provided. This method comprises depositing, such as by sputtering, a uniform layer of an electrically conductive metal onto all or a portion of a surface of a substrate. In an embodiment, the electrically conductive metal is pure molybdenum (Mo) or is MoS₂. In an embodiment, the substrate is a flexible glass sheet. Subsequent to deposition of the electrically conductive metal, a pattern of leads and/or contacts is created by removing regions of the electrically conductive metal. That is, electrically conductive metal is removed from the substrate other than where a lead and/or contact is positioned. In an embodiment, the layer of electrically conductive metal is between about 35-100 nm, 35-75 nm, 35-60 nm, 40-60 nm or about 50 nm. In an embodiment, the deposition is conducted under vacuum. In an embodiment, portions of the electrically conductive metal are removed by ablating except for a pattern of leads and/or contacts on the layer; the pattern can be specified, for example, in an electronic drawing file transferred to instrument, such as a laser micromachining or a high speed scribing laser technique that comprises a laser beam. The laser beam is scanned across the electrically conductive metal to produce the required pattern of leads and contacts, and also creating a gap positioned in the leads. In embodiments, the sensor leads and/or contacts are separated by a gap of about at least about 0.08 mm, 0.09 mm, or 0.1 mm to about 2.25 mm, 2.1 mm, or 2.0 mm. MoS₂ is deposited in the gaps, where sensors or a ‘sensing region’ will be created in a later step. The method produces, in some embodiments, a glass sheet coated with 3-8 atomic layers of semiconducting MoS₂ that is laser-patterned to form leads and contacts, as part of the sensor chip. In certain embodiments, the laser removes the Mo or MoS₂ that is not needed, retaining a layer of MoS₂ only at desired locations, such as in the gap between leads, leads, and/or contacts. In embodiments, more than one laser and/or more than one type of laser is used in the method.

In some embodiments, the method may further comprise treating the sensor chip to anneal or crystalize the MoS₂, thereby transforming the amorphous MoS₂ into a crystalline two-dimensional transducer. In an embodiment, the treating comprises one or both of exposing the sensor chip to a laser to transform a portion of amorphous MoS₂ into crystalline MoS₂ and heating the sensor chip to a temperature of between about 275-550° C. or between about 300-500° C. In an embodiment, the heating is performed for between about 2-20 minutes, 2-15 minutes, 5-15 minutes, 6-12 minutes or 8-12 minutes. The method may further comprise functionalizing the sensor chip by depositing a reagent, an assay specific reagent, or a mixture of assay specific reagents onto the sensor, thereby depositing one or more reagents with binding specificity for one or more analytes of interest. In an embodiment, after functionalizing with the reagent(s), the sensor chip is dried to remove solvent present in the solution used to deposit the reagent. In an embodiment, the sensor chip is dried at a temperature of between about 25-45° C., or between 30-45° C. for less than about 20 minutes, 15 minutes, 12 minutes or 10 minutes.

In an embodiment, the reagent, the assay specific reagent, or the mixture of assay specific reagents comprises a reagent, such as a rFAB binding member for an analyte of interest, in a solvent. In an embodiment, the solvent is an aqueous solution, optionally pH adjusted. In an embodiment, the solvent or aqueous solution is pH adjusted based on the isoelectric point of the rFAB such that the rFAB has little to no net electrical charge. In an embodiment, the pH of the solvent or aqueous solution is adjusted to be below the isoelectric point of the rFAB so that the rFAB carries a net positive charge. In an embodiment, the pH of the solvent or aqueous solution is adjusted to be above the isoelectric point of the rFAB so that the rFAB carries a net negative charge.

In yet another aspect, a method of detecting one or more analytes of interest in a subject using any of the biosensor chips or biosensor systems described herein is contemplated. This method may comprise collecting, providing or receiving a sample, such as a nasal, throat, blood, urine or other sample from the subject, and optionally contacting the sample with a processing reagent and/or an extraction reagent, and depositing the sample or the processed sample onto a cassette that comprises a sensor chip. Before or after the depositing, the test cassette is inserted into a reader. The reader is configured to determine a test result and to transmit the test result to a receiving member or means, which may be read by a user of the system. By reading the test results, which are indicative of presence or absence of the one or more analytes of interest in sample from the subject, the user can determine whether or not the subject has one or more analytes of interest. In some embodiments, the test results may be transmitted to the receiving means in less than about 5 minutes, 4 minutes, 3 minutes, 2 minutes, or 1 minute. It will be appreciated that the method is not restricted to samples from a subject (living or deceased, human or non-human), and is equally applicable to samples from water, soil, food, fluids, waste streams, waste deposits.

In addition to the exemplary aspects and embodiments described above, further aspects and embodiments will become apparent by reference to the drawings and by study of the following descriptions.

Additional embodiments of the present compositions and methods will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

The details of one or more embodiments of the disclosure are set forth in the accompanying drawings/tables and the description below. Other features, objects, and advantages of the disclosure will be apparent from the drawings/tables and detailed description, and from the claims.

FIGS. 1A-1B illustrate embodiments of a sensor chip.

FIGS. 2A-2D are illustrations of an embodiment of a sensor chip and cassette (FIGS. 2A-2B) and a system comprised of a sensor chip, cassette and reader (FIGS. 2C-2D).

FIG. 3 is an embodiment of a system comprised of a sensor chip, cassette and reader, where the sample port is on the reader.

FIG. 4 shows an embodiment of a system comprised of a sensor chip, cassette and reader.

FIG. 5 is a flow chart showing an embodiment of a sequence of events for use of the system.

FIGS. 6A-6B are a flow chart (FIG. 6A) and illustrations (FIG. 6B) of an embodiment of sequence of events for use of the system.

FIG. 7 illustrates a high-throughput process for production of sensor chips.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The magnitude of change in the examples is not meant to be rate limiting. The meaningful change will depend on the specific diagnostic assay. A meaningful change to inform a treatment decision may be a change from baseline, a change from matched controls, a change from a reference value from a healthy individual, a change from a non-involved area or some other change. The change may be more or less than 1%, 10%, 25% 50%, 75%, 90% or 99%. The change may be based on an average, a standard deviation, a cutoff, an interquartile range or some other meaningful change. The changes may be based on meaningful changes in a database, a clinical trial, previous experiments or intra-experimental controls. The changes may be based on high or low values. The changes may or may not be statistically significant.

### II. Testing System

In one aspect, a system comprised of a sensor chip and a reader is provided. In embodiments, the system comprises a sensor chip, a reader, and a software application. The sensor chip, in embodiments, is within a cartridge, housing or cassette, to ease handling by a user. The system may optionally comprise a software application downloadable to a user's electronic device, the software application having instructions for evaluating signal received from the reader to determine presence or absence of an analyte of interest in a sample placed on the sensor chip or its optional cassette. These components of the system are now described.

### A. Sensor Chip

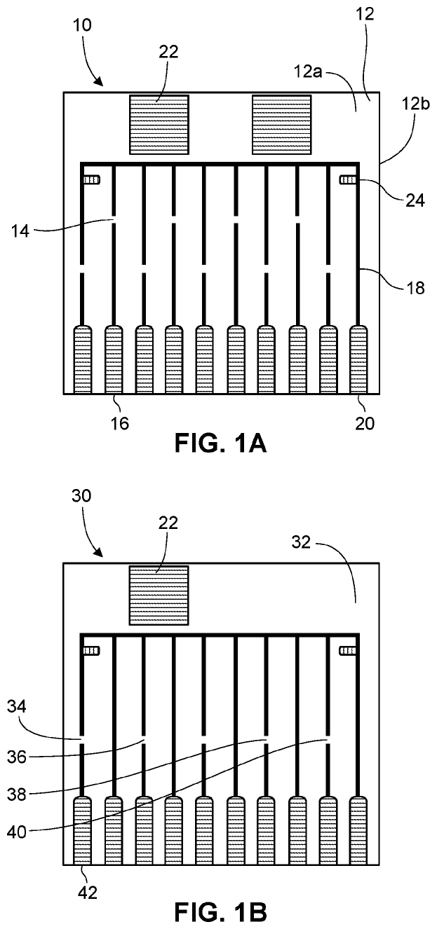

An embodiment of a sensor chip is shown in FIG. 1A. Sensor chip 10 is comprised of a substrate 12 that has a first or upper surface 12a and a second or lower surface 12b. The first surface of the substrate comprises an array of or a plurality of sensing regions. In the illustrated embodiment, sensor chip 10 comprises nine sensing regions, such as sensing region 14 which is exemplary. Each sensing region is in a gap between electrical leads that extend to an electrical contact, such as electrical contact 16 which is exemplary of the ten electrical contacts in sensor chip 10. In an embodiment, one or more of the electrical contacts is a ground lead, such as lead 18 in sensor chip 10 which does not have a sensing region within the lead extending from contact 20. The electrical contacts extend essentially to the edge of the substrate, for contact with corresponding contacts in a separate reader that is configured to receive the sensor chip, or in embodiments, a cassette in which one or more sensor chips is contained or secured. The reader is described elsewhere herein and, in embodiments, comprises components and/or circuitry to provide electrical current to the electrical contacts of the sensor chip, to detect a change in resistance or conductance in one or more, or in each, sensing region of the sensor chip, and/or to transmit a signal corresponding to the detected change or to a processed signal corresponding to the detected change.

In embodiments, the substrate has a first planar surface and a second or opposing planar surface. The substrate is generally of any material to provide a suitable surface roughness and which can be used as a support or base for receiving on one surface thereof materials for the sensor, such as the transition metals and detection agents described herein. In an embodiment, a substrate described herein has an upper surface comparable to the roughness of a silicon wafer used in a semiconductor application when measured using the same technique. In an embodiment, the surface roughness is measured with a regular atomic force microscopy probe and, in embodiments, the measured roughness is less than about 2 nm or less than about 1 nm. An ordinarily skilled person will recognize that surface finish (i.e., roughness) can be measured using various methods. It is understood that the parameter Ra is the universal, most widely used parameter for roughness internationally. Ra is the arithmetic mean of the departure of a surface's roughness profile from a mean line (i.e., a reference line representing the overall surface). A hypothetical substrate's surface roughness of 10 nm means that such a substrate has a mean (average) departure of 10 nm from its overall surface, as measured across the entire surface of the substrate. In an embodiment, a substrate has an upper surface having a surface roughness of about 0-100 nm, or about 0-75 nm, or about 0-50 nm, or about 0-25 nm, or about 0-10 nm. In embodiments, the upper surface has a roughness comparable or equivalent or essentially equivalent to the roughness of a silicon wafer which is suitable for semiconductor production. The substrate can have any shape, for example the substrate may be provided as a planar substrate, though the substrate can have any useful shape or configuration.

Sensor chip 10 also comprises an identifier or symbol, such as identifier 22. Identifier 22 can take the form of, for example, a universal product code, an electronic product code, a serial number, a bar code, a two-dimensional quick response (QR) code, or the like. The identifier embeds information that is conveyed or ascertained by the reader and/or a user of the sensor chip, that information including, for example, what analyte(s) of interest or disease(s) or disorder(s) the sensor chip will detect by determining presence or absence of one or more analyte(s) of interest. The identifier can also include information regarding the sensor chip manufacturing lot, manufacturing date, expiration date, and/or sensor chip identification number. In an embodiment, the identifier is readable by the reader and/or a user's electronic device, and conveys information regarding an algorithm and/or a software application to be launched. The algorithm and/or software application can include instructions for the reader and/or the user's electronic device to conduct the assay, including for example when to place a sample on the sensor chip or the cassette holding the sensor chip, when to insert the sensor chip or a cassette holding the sensor chip into the reader, when to supply electricity to the contacts on the sensor chip, when to interrogate for a change in resistance, and/or when the assay is completed. The algorithm can include steps for mathematical manipulation of signal corresponding to resistance or conductivity of each sensing region, for example, to normalize signal obtained when sample is present in a sensing region against signal for the sensing region when no sample is present, to account for or correct for background resistance (or conductance) of a sensing region. The sensor chip 10 can also include one or more fiducials, such as fiducial 24. A fiducial can be useful, for example, during manufacture of the sensor chip to find, for example, the sensing regions along each electrical lead.

FIG. 1B illustrates another embodiment of a sensor chip. Sensor chip 30 is comprised of a substrate 32 that has a first or upper surface and a second or lower surface. The first surface of the substrate comprises an array of or a plurality of sensing regions. In the illustrated embodiment, sensor chip 30 comprises four sensing regions, such as sensing regions 34, 36, 38 and 40. Each sensing region is in a gap between electrical leads that extend to an electrical contact, such as electrical contact 42 which is exemplary of the electrical contacts in sensor chip 30. In this embodiment, the sensor chip comprises one ground lead per sensing region. Other configurations are contemplated, for example, one ground per two sensing regions or one ground per four sensing regions.

In an embodiment, each sensing region has a laser-etched gap or line around all or a portion of the region, to isolate each sensing region from adjacent sensing regions. As mentioned, each sensing region is composed of a transition metal, and typically is composed of a transition metal and an element (such as nitrogen, sulfur, selenium, phosphorous, hydrogen or carbon). The transition metal-element compound is rendered semiconductive or conductive by treating it with heat or with a laser. In an embodiment, the transition metal-element compound is MoS₂. MoS₂ untreated by heat or laser exposure is amorphous, and might exhibit some electrical conductance. Amorphous MoS₂ treated by heat or laser becomes crystalline and semiconductive or conductive. A laser-etched gap or line around a semiconductive or conductive MoS₂ sensing region electrically isolates each sensing region from the other.

The dimensions of the sensor chip may change depending on the number of sensing regions, number of ground leads, number of control sensing regions, and the like. For example, a sensor chip with detection reagents for four analytes of interest, for example, influenza A, influenza B, respiratory syncytial virus, and SARS-COV-2, will comprise a sensing region for each analyte of interest and at least one negative control sensing region, for a total of five sensing regions. Each sensing region is in a gap of an electrical lead, thus the sensor chip has at least five electrical leads. Depending on the number of ground leads, the sensor chip of this illustrative embodiment for detection of four different analytes of interest can have between six and ten electrical leads.

In an embodiment, a sensor chip is secured within a cartridge, housing or a cassette, to ease handling of the sensor chip by user. The cartridge, housing or cassette, which will be referred to hereafter as a cassette, can be a disposable single-use cassette or a reusable cassette, in either case made typically made of a plastic material. The cassette can have a depression, a slot or a port into which the sensor chip can be placed or inserted. In embodiment, the cassette and/or sensor chip is/are configured so that the sensor chip fits in the cassette only in one orientation. In an embodiment, the cassette for the sensor chip is dimensioned similar to a credit card. In an embodiment, the cassette can have an upper and lower member that securely fit together, such as via a snap fit, and the sensor chip is positioned between the upper and lower members. In an embodiment, the cassette can have a well or port dimensioned to receive a sample and to communicate the sample to the sensor chip. In an embodiment, a channel extends from the well or port to the sensor chip, for fluid communication between the well or port and one or more, or each, sensing region on the sensor chip. In an embodiment, the well or port is positioned on the cassette such that once the cassette is inserted into the reader, the well or port remains accessible for receipt of a sample.

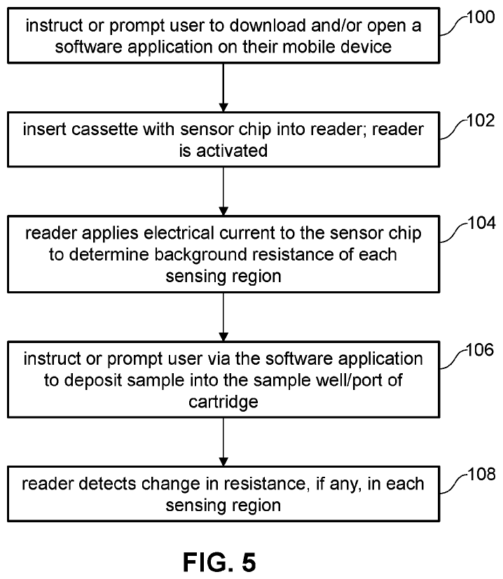

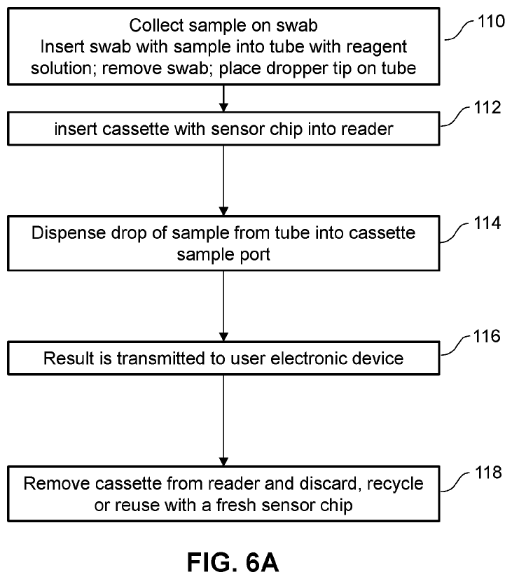

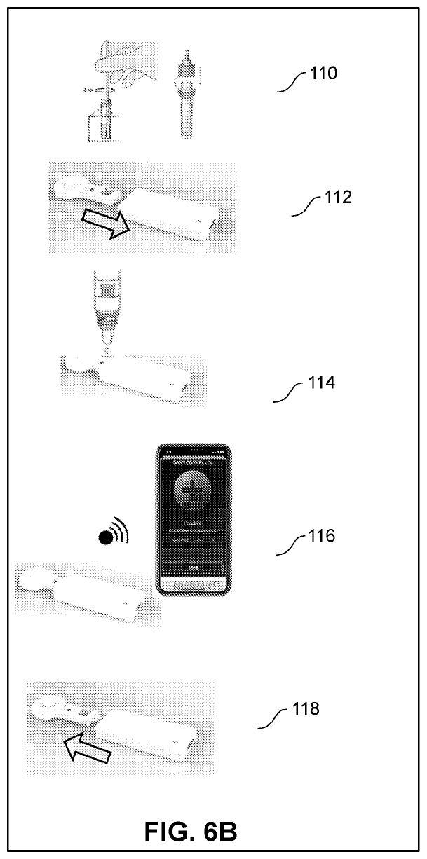

In an embodiment, the cassette is dimensioned so that the electrical contacts of a sensor chip inserted or held in the cassette are exposed for contact with components and/or circuitry in the reader, in order to provide electrical current to the electrical contacts of the sensor chip and/or detect a change in resistance in a sensing region between electrical leads. In another embodiment, the cassette is dimensioned and/or configured so that it fits in a reader in only or solely one orientation. In an embodiment, the cassette and/or reader are configured so that insertion of the cassette into the reader activates the reader, e.g., powers on the reader, to initiate a series of events, described infra with reference to FIGS. 5-6. In embodiments, the cassette is dimensioned so that the sensor chip with inserted or placed in or on the cassette has a corresponding set of electrical contacts and/or leads that match and/or operably connect with the sensor chip. In other embodiments, the cassette has a set of electrical leads and/or contacts for operable connection with a resistance detection device capable of collecting current or resistance readings, for transmitting to a software application for analysis. In embodiments, the cassette comprises an identifier or a symbol, such as those described elsewhere herein. The cassette may also comprise a location, such as a depression or region, to hold a desiccant.

### A1. Detection Agents

The sensing region(s) on a sensor chip comprise a detection agent that interacts with an analyte of interest. The detection agent, also referred to herein as a reagent or an assay specific reagent, is deposited onto the sensing region(s) of a sensor chip, to “functionalize” the sensor chip for detection of one or more analytes of interest. The detection agent is capable of binding, directly or indirectly, to an analyte of interest. The binding can be covalent or non-covalent, such as electrostatic interactions, hydrogen bonds, van der Walls forces, hydrophobic interactions, weak chemical interactions, and the like. In embodiments, the detection agent is a protein, a polypeptide, an oligopeptide, a peptide, a nucleic acid, an oligosaccharide, or a polysaccharide. Some examples are provided, but are merely exemplary of the possibilities.

In an embodiment, the detection agent is an antibody, an antibody fragment, or a peptide. In an embodiment, the peptide has between about 2-400 amino acid residues or between about 20-200 amino acid restudies. The antibody or antibody fragment, in an embodiment, is a monoclonal antibody (mAbs) or mAb fragment, which can be expressed recombinantly. In some embodiments, the recombinantly expressed mAb fragment is an antigen binding fragment (FAB) (rFABs). In embodiments, the rFAB is a F(ab′)₂ fragment, an Fab′ fragment, a Fab fragment, or an Fv fragment. In an embodiment, a antibody fragment is prepared by enzymatic cleavage. In an embodiment, the detection agent is a single domain antibody fragment (sdAb). sdAbs occur naturally in camelids, or can be derived from V_{H} or V_{L} variable regions of a mAb or a heavy-chain-only antibody, or from antibodies lacking a CH1 domain (Ig-NAR).

The detection agent can also be a nucleic acid. The nucleic acid can be a DNA or RNA sequence, and will be complementary to all or a portion of the analyte of interest.

In embodiments, the detection agent is attached to a linker. The linker can be attached to the detection agent in a chemical process separate from preparation or creation of the detection agent. The linker can also be attached to the detection agent during the preparation or creation of the detection agent. For example, a linker can be chemically reacted with a chemical moiety on an amino acid residue of an antibody fragment or peptide detection agent. A linker can be expressed as part of an antibody, antibody fragment, or peptide. For example, a peptide or protein detection agent can be recombinantly expressed as a fusion peptide or fusion protein comprised of a detection agent portion and a linker portion. In an embodiment, the linker portion is at the C-terminal or N-terminal end of the peptide or protein.

In embodiments, the linker is an amino acid linker with between 2-30, 2-25, 2-20, 2-16, 3-30, 3-25, 3-20, 3-16, 4-30, 4-25, 4-20, 4-16, 5-30, 5-25, 5-20, 5-16, 6-30, 6-25, 6-20, 6-16, 7-30, 7-25, 7-20, 7-16, 8-30, 8-25, 8-20, 8-16, 9-30, 9-25, 9-20, 10-30, 10-25, 10-20, or 10-16 amino acid residues. Examples include dipeptide pairs that are non-helical, such as Pro-Pro, Trp-Trp, Met-His, Gln-Pro and Pro-Leu, or pairs that are helical, such as Cys-Met, Arg-Met, Arg-Lys, Gln-Arg and His-Arg. Other linker examples are (Gly-Gly) n, and (Lys-Lys) n, where n is 1, 2, 3, 4 or 6, Gly-Gly-Gly-Gly-Ser (SEQ ID NO: 2). Another linker example is HLLQPTQNPFRN (SEQ ID NO: 1, also referred to as “HLL”) or a sequence with 90%, 80% or 70% sequence identity to SEQ ID NO: 1.

In other embodiments, the sensing region comprises a generic member or generic linker. The detection agent is prepared to include a member that bind the generic member or generic linker. Examples of this include biotin-avidin (AviTag) streptavidin-biotin, digoxin-anti-digoxin, and small molecule-synthetic ligand binding pairs, such as the haloalkane dehalogenase that binds a synthetic ligand (HaloTag).

In some embodiments, the detection agent (also referred to herein as a reagent or a biomolecule) is a component of a mixture of assay specific reagents that is deposited on the sensor chip. The mixture comprising the detection agent can include, for example, a solvent, a salt, a buffer, a sugar, a preservative, and other agents. The solvent can be an organic solvent, an aqueous solvent mixture, water, or a buffer.

In some embodiments, the analyte of interest is an infectious agent. In an embodiment, the infectious agent is a virus, a bacteria, or a fungus. In other embodiments, the analyte of interest is a drug, such as a therapeutic drug or a drug of abuse. In embodiments, the analyte of interest is an antibody, a protein, or a peptide.

In other embodiment, the sensor chip in the test cassette is configured for detection of one or more analytes of interest, such as one or more analytes of interested selected from a nucleic acid, an antibody, a protein, a peptide, a therapeutic agent, or a drug of abuse. In embodiments, the nucleic acid is a DNA or an RNA sequence.

In some embodiments, the mixture of assay specific reagents may comprise monoclonal antibodies (mAbs) expressed recombinantly in the form of recombinantly expressed FABs (rFABs). In certain embodiments, each rFAB may comprise a linker, such as HLL. The linker selectively binds to the transition metal, such as MoS2, in the sensing region of the sensor chip. In some embodiments, each rFAB may be specific for an infectious agent and the sensor chip may comprise multiple sensing regions that comprise different detection agents, such as rFABs, specific for different analytes of interest. Alternatively, the sensor chip may comprise multiple sensing regions that comprise different detection agents specific for different regions of the same analyte of interest.

The system disclosed herein is capable of detecting multiple analytes (e.g., infectious agents) at the same time. In certain embodiments, users can test for 1, 2, 3, 4, 5, 6, 7, 8, 9, 10, 11, 12, 13, 14, 15, 16, 17, 18, 19, 20, 25, 30, 35, 40, 45, 50 or more analytes of interest from a single sample applied to a test cartridge comprising a sensor chip using the system disclosed herein. Depending on the end goal, the chip design can have as few as one sensor or as many sensors as a multianalyte design requires.

In an embodiment, a sensor chip with at least about 3, 4, 5, 6, 7, 8, 9, 10, 11 or 12 sensing regions per sensor chip is provided. Each sensor chip, in embodiments, is functionalized with a detection agent that binds directly or indirectly an analyte of interest and/or is functionalized with a detection agent or other reagent to serve as an assay control. In an embodiment, each functionalized sensor chip provides a reproducible test results with an analytical panel showing a limit of detection of between 0.01 and 10 pg/mL.

In some embodiments, the reagent, or the mixture of assay specific reagents, comprises a monoclonal antibody (mAb) expressed recombinantly in the form of recombinantly expressed antigen binding fragment (FAB) (rFABs). In embodiments, the rFAB is a F(ab′)₂ fragment, an Fab′ fragment, a Fab fragment, or an Fv fragment. In certain embodiments, the rFAB may comprise a linker peptide sequence, such as the linker peptide sequence HLLQPTQNPFRN (SEQ ID NO: 1). In some embodiments, each rFAB may be specific for an analyte of interest, such as an infectious agent, and the sensor chip may comprise multiple sensors that are deposited or spotted on the sensor chip surface with different rFABs specific for detection, if present, of different analytes of interest. In embodiments, one or more rFABs deposited or spotted on the sensor chip surface is a recombinantly expressed fusion protein of a linker peptide, such as SEQ ID NO: 1 or a linker peptide with 90%, 80% or 70% sequence identity to SEQ ID NO: 1.

In an embodiment, the detection agent is attached or immobilized to the sensing region of a sensor chip. Interaction of the detection agent with an analyte of interest brings the analyte of interest closer to the surface of the sensing region, causing a charge and/or conductance/resistance alteration in the sensing region. Many analytes of interest, particularly protein analytes of interest, carry a positive charge or a negative charge. Other analytes of interest with a nearly neutral or neutral charge, such as some proteins and some carbohydrates (e.g, fungal mannans) and some polysaccharides. In an embodiment, the detection agent is modified to have or selected based on it having a positively charged moiety or negatively charged moiety. Addition or modification of the detection agent to have a positive charge or a negative charge is contemplated as an approach to enhance sensitivity of the sensor chip. Binding of the detection agent with an analyte of interest, where the detection agent has been modified to include a positively charged moiety or negatively charged moiety will increase avidity of the binding event and thus bring the analyte of interest closer to the sensor surface, to improve detectability. In embodiments, the charge type and/or density of charged moieties on the detection agent is considered, and modified as needed.

In an embodiment, the detection agent is capable of detecting an antibody that interacts and/or binds with the analyte of interest. For example, a first antibody is mixed with the sample and the sensor chip comprises a detection agent that binds the first antibody when it is bound to the analyte of interest. In another embodiment, a charged amino acid residue or end cap is present on a terminal end of one or both heavy chains of the detection agent, to add either a position or a negative charge to the detection agent. In an embodiments, the charge on the detection agent is the same as the charge of an analyte of interest under the assay conditions, so that the detection agent adds additional same charge to the sensing region. In another embodiment, a charged nanoparticle is mixed with the sample.

In another embodiment, a reagent is included in conjunction with the detection agent to add, alter or replace charge density or type. For example, positively charged or negatively charged microbeads or nanobeads can be deposited in combination with the detection agent. This approach offers an way to control density of charge in the sensing region, and an array of chemistry techniques can be applied to the bead to add charge moieties and to bind them to the detection agent, if desired.

In embodiments, a protein based binder or linker with a positive charge or a negative charge can be recombinantly expressed as a fusion protein with the expression of the detection agent. In other embodiments, a non-protein based binder or linker with a positive charge or a negative charge can be recombinantly expressed as a fusion protein with the expression of the detection agent. The non-protein binder can be conjugated to a nanobead or a microbead, if desired.

In some embodiments, the sensor chip comprises as a detection agent a nucleotide, peptide, aptamer, antibody fragment, or antibody from any of a variety of animal species. Binding of an analyte of interest to the detection agent is evidenced by alteration in resistance (or conductance) of electrical current across the sensing region, and the alteration is detectable with exquisite sensitivity to permit, for example, detection of an analyte of interest present at concentrations in the sample as low as 10 fg/mL. In some embodiments, the detection agent is attached to the sensing region via a linker, such as an amino acid linker. In other embodiments, the detection agent is an antibody, such as a monoclonal antibody and/or a Fab fragment of the same, and the sensor is able to detect 1 pg/mL of a target nucleoprotein analyte of interest. As can be appreciated, the sensors provide an approach to detect an analyte of interest present in a sample at concentrations that are 1,000 fold or 10,000 fold lower than can be detected using a conventional immunoassay detection approach.

In an embodiment, the substrate of the sensor chip is processed or treated to yield a surface with a specified roughness or smoothness or flatness. In an embodiment, the surface has a roughness comparable to a polished silicon wafer. In other embodiments, the surface of the substrate, prior to deposition of a detection agent, is treated with a laser or with heat, to melt a surface layer to reduce the peaks and valleys on the surface. In an embodiment, the surface of the substrate, prior to deposition of a detection agent, is treated with a laser to cause divots or holes in the surface.

### B. Reader

The system comprises a reader that is configured for interaction with the sensor chip and, optionally, with a software application downloadable to or downloaded onto a mobile device. Generally, the reader comprises a port, opening or slot dimensioned and/or configured to receive a cassette comprising a sensor chip. Alternatively, the reader can comprise a port, opening or slot dimensions for receipt of a sensor chip. The reader also comprises circuitry to provide an electric current to the sensor chip and a sensor to evaluate resistance in the sensing region(s) of a sensor chip.

In an embodiment, the reader comprises a transmitter or a transceiver to send, or send and receive, signal based on the resistance of the sensing region(s) to an external electronic device. The system also comprises a software application downloadable to or downloaded onto an electronic device, such as a user's mobile device, comprising a processor operably coupled to a memory that includes instructions stored thereon for interfacing with the reader, as well as an additional storage component. The software application comprises an algorithm for processing signal from the sensor chip. The reader and the electronic device are operably coupled such that signal and/or data originating from the sensing regions of the sensor chip and/or the reader are accessible to the electronic device.

In some embodiments, the reader may be a handheld reader that is operated by battery, whether replaceable or rechargeable. The size of the reader can be dimension to be similar to, for example, a thumb drive. The reader is dimensioned to receive a sensor chip or a cassette with a sensor chip. The reader may have a light indicator to show on/off. The reader can be configured to be insertable into a computer, such as into a USB or USB-C port or via a cable. The reader comprises a transmitting member or means, for transmission or communication of a result from the test cartridge and/or sensor chip to a user or to a receiving member or means. The transmitting member or means can be a wireless technology, such as a short range wireless technology (e.g., Bluetooth®) or a wireless adapter for transmission via WiFi, or a physical or wired technology, such as a USB, USB-C or other physical connection. In some embodiments, the receiving member or means may be a smartphone or other mobile device, and the test result may be transmitted and/or received via an app or software application on the smartphone or mobile device.

In an embodiment, the circuitry in the reader comprises one or more of a microprocessor, leads and contacts, a transmitter and/or receiver, one or more indicators, a battery, and/or a transducer.

In certain embodiments, the testing system is configured such that the cassette comprising one or more sensor chips is inserted into reader. The reader, in embodiments, is configured to power on when it receives a test cassette. Once the reader is powered on, whether automatically by insertion of the test cassette or via an on/off user active switch, the reader is programmed to initiate and execute a series of events. In an embodiment, a first event is to obtain a signal from the sensor chip, to determine a baseline or background signal from that particular sensor chip. The signal is generally a resistance signal, however it will be appreciated that a conductance signal can also be used. In an embodiment, another event is to determine if a change in the measured resistance (or conductance) signal occurs. In an embodiment, another event is to initiate a timer for a defined time period to determine resistance signal of the sensor chip. In an embodiment, another event is to determine resistance signal during or after the defined period. In an embodiment, another event is to scan an identifier or symbol on the sensor chip and/or the test cassette. In an embodiment, another event is to transmit measured resistance signal, measured background resistance signal, information connected to the identifier or symbol, or any combination thereof to a user or a user's receiving means or any other designated receiving member. In an embodiment, the reader and/or the receiving means or member comprises an algorithm that is capable of processing the signal, such as by taking an average, median or mean of the measured resistance signal, or normalizing a measured resistance signal against the measured background resistance signal, and/or processing the measured resistance signals based on information in the identifier or symbol. In a model embodiment, a system is configured such that insertion of a test cassette into the reader initiates the reader to obtain a background resistance, to interrogate the sensor chip in the test cassette at defined intervals for a defined period of time (e.g., every 5 seconds for 2-3 minutes) until a change in resistance is detected. The change in resistance will correspond to a change in the electrical conductivity of the sensor due to the resistance changes from the selective binding of an analyte of interest in the sample, which is place on the test cartridge after or before insertion into the reader. The reader will collect resistance data for a defined period of time, optionally store the data, and transmit it to a mobile device or workstation or server. In an embodiment, the mobile device or workstation or server has a memory that stores instructions for processing the data, typically in the form of an app, and a processor to execute the instructions. The data received by the mobile device, workstation or server is thus analyzed to determine or generate a result, which optionally can be displayed on the mobile device, workstation or server and/or further transmitted or communicated.

In an embodiment, the reader is configured to and comprises the components to obtain a resistance reading, such as by a resistance detection device, and to transmit the reading or data to a software application on a user's electronic device. A resistance detection device can collect current or resistance readings from a sensor chip using either a cable plugged into the user's electronic device or a wireless connection and can transmit the readings or data to a receiving member (also referred to herein as a receiving device), such as electronic device, that can have a specific application to receive and manipulate the readings or data. In an embodiment, the reader is capable of two-way communication, to send and to receive, and the specific application on the receiving member is configured to signal to the reader that resistance readings or data, or a final test result in embodiments where the reader is configured to analyze the resistance readings or data, has been received and/or for the receiving member to provide an update to the firmware in the reader.

In an embodiment, the reader has one or more slots to receive a cassette or sensor chip. The reader can plug directly into an external electronic device, e.g., a user's electronic device that can be a mobile device or a computer. In this embodiment, the reader is operably connected to the external electronic device for example via USB, USB-C, or a cable, which will supply current to the reader and will receive data from the reader. In embodiments where the reader is wireless and not configured to plug into or be connected to a user's external electronic device, the reader can have a rechargeable battery.

In an embodiment, the reader comprises one or more indicators. In an embodiment, the indicator is a visual indicator, such as one or more indicator lights. In an embodiment, the one or more indicators is an audible signal, such as a sound (beep, ding, chime, etc.) or a sensible (sensory) signal, such as a haptic or a vibration. In embodiments, the one or more indicators inform a user of status of events prior to, during and/or subsequent to conducting an assay, such as the status of an assay being run, completion of an assay, status of data transmission to an external receiving member (e.g., the user's electronic device, including success of transmission, failure of transmission, and/or transmission in progress, proper insertion of a sensor chip and/or a cassette into the reader, detection of a sample onto the sample well or sensor chip, initiation of an assay, transmission of data, and/or low batter of the reader. When the indicator is a light, a light can turn on, turn off, change color, blink or flash to inform a user of any one or any combination of these events. When the indicator is an audible signal, a signal can be given to inform a user of any one or any combination of these events. The indicator can also be a sensory signal, such as a haptic or vibration, on the system or on the user's electronic device to indicate any one or any combination of these events. It will be appreciated that any combination of indicator lights, audible signals and/or sensory signals can be used.

As mentioned above, in an embodiment, the reader is configured to scan an inserted cassette or sensor chip for an identifier or symbol on the sensor chip and/or the cassette. That is, once the cassette or sensor chip is inserted into the reader, it is programmed to look for an identifier or symbol, which can be a barcode, an RFID chip, a QR code, a magnetic strip, etc., that informs the reader of information regarding the sensor chip, including the assay (e.g., the analyte(s) of interest the sensor chip is capable of detecting), to identify an assay specific file stored on the reader and/or on an external electronic device in order to initiate a sequence of events for detection of an analyte of interest in a sample.

In an embodiment, a change in resistance detected by the reader or an App corresponds to an instruction to the reader that a sample has been applied. Thereafter, the reader can take a resistance readings every 5 to 10 seconds for a time period of between about 90 seconds to 5 minutes, depending on the assay. A resistance detection device can monitor the change in resistance or current and report the data to an external electronic device where an algorithm in the App analyzes the data and reports a test result. The test results can be qualitative, semi-quantitative or quantitative.

In an embodiment, the sensor chip is a component of a system designed to enable use of the sensor chip at home in other locations like a doctor's office. The system includes a reader or transmitter (or a reader with a transmitter). In embodiments, the reader or transmitter has dimensions similar to a typical USB drive device. The reader or transmitter can be powered by line or battery (disposable or rechargeable) power sources. In an embodiment, the reader or transmitter supplies electrical current to monitor resistance of, in, or on one or more of the sensing regions of a sensor chip that occurs during an assay conducted in one or more of the sensing regions of a sensor chip. In an embodiment, the reader or transmitter collects the resistance measurements and reports the measurements to a receiving device (also referred to herein as a receiving member), which can be an external electronic device such as a user's mobile device. In embodiments, the reader or transmitter collects resistance measurements from an assay and transmits the data by to a receiver or receiving device, such as a smart phone, laptop computer or designed for purpose result receiver/analysis device. The receiver device collects the transmitted data for analysis via an analysis algorithm of any design and/or functionality to convert the transmitted data for resistance into an assay result indicating presence or absence of an analyte of interest, the result being either qualitative or quantitative.

In an embodiment, the sensor chip is configured to determine presence or absence of an analyte of interest that is a nucleic acid, and the reader can include a component to provide heat to the sensor chip. In an embodiment, the component is positioned and dimensioned to change the temperature of the sensing region(s), typically by providing heat to raise the temperature, to approximately 80%, 85%, 90%, 95% or essentially 100% of the surface area of the sensor chip. In an embodiment, the component to provide heat is able to heat the sensor chip in the presence of a sample deposited thereon to between about 40° C. to 85° C. with an accuracy of approximately +/−1° C. In an embodiment, a thermistor is positioned in the cassette to measure temperature of the sensor chip, and to communicate temperature to the reader. The reader comprises circuitry to increase and/or decrease current to the component to provide heat in accord with the temperature readings from the thermistor and an assay algorithm stored in memory of the reader, or communicated to the reader from the receiving device. In an embodiment, the component to provide heat maintains a constant temperature or raises the temperature. A heat sink can be present in the cassette or adjacent the substrate of the sensor chip to decrease temperature, if temperature cycling is desired. In an embodiment, flow of current from the reader to the component to provide heat is initiated prior to adding sample to the sample port on the cassette. In an embodiment, when a preselected temperature is reached on the sensor chip, the user is instructed to deposit sample on the cassette.

In embodiments, the reader or transmitter comprises a QR coder reader, RFID chip reader, or a camera to “read” an indicator or symbol disposed on the test cassette and/or the sensor chip. The test cassette is configured to hold, receive and/or secure the sensor chip. The test cassette, in embodiments, comprises electronic contacts to connect to electric current flow for a source typically in the reader or transmitter, and may also comprise a computer chip to direct the current in sequence from one lead on the sensor chip to the next in a repetitive motion for a period of time. In embodiments, the period of time corresponds to the period of time for an assay to be conducted. During this period of time, the reader or transmitter is collecting resistance data associated with the current flow in the sensing regions of the sensor chip, and records the data in a memory device within the reader or transmitter for later or immediate transmission to the receiving device. In an embodiment, the reader or transmitter comprises a memory and a processor, and an algorithm stored in memory for analysis of the resistance data to determine an assay result indicating presence or absence of an analyte of interest, the result being either qualitative or quantitative, and the assay result is transmitted to a receiving device.

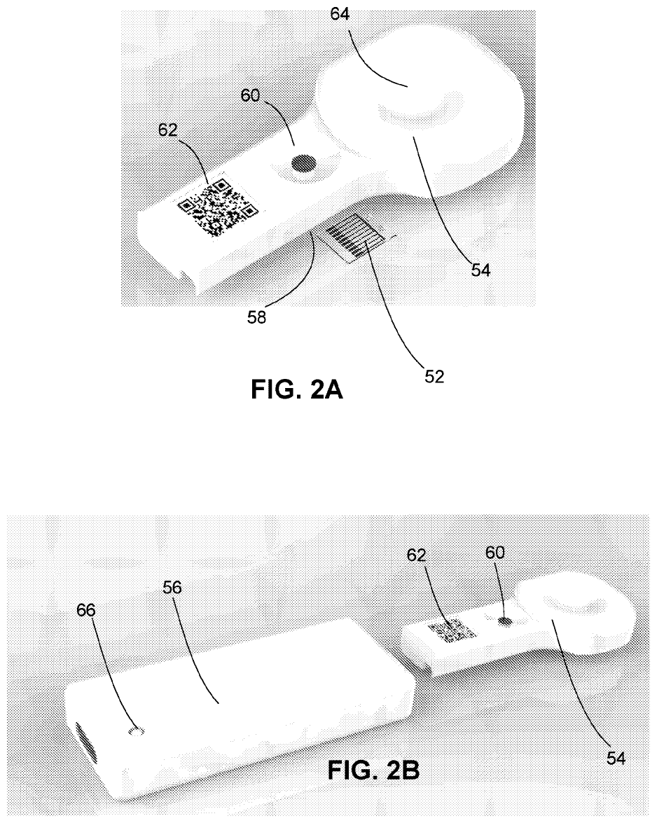

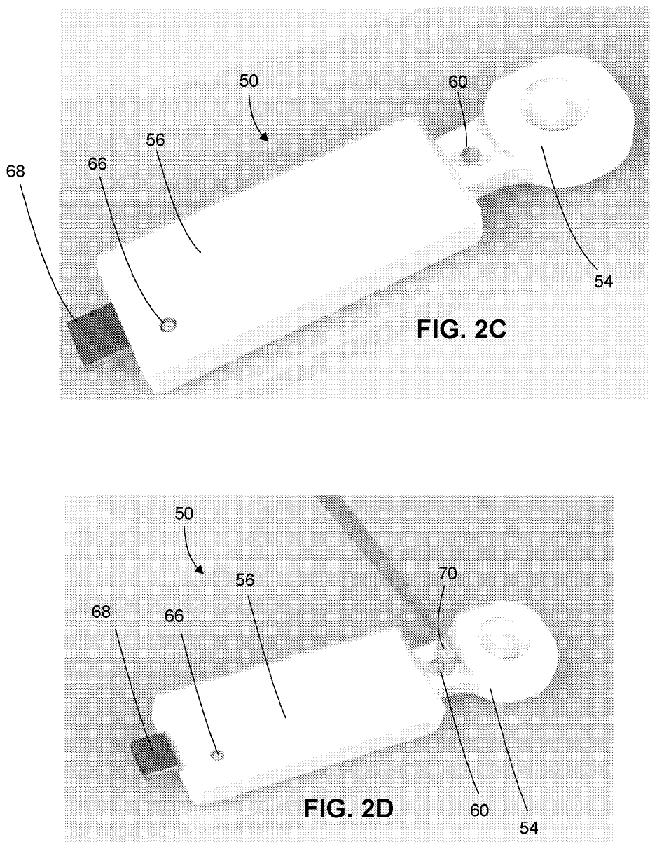

Exemplary embodiments of the system are illustrated in FIGS. 2-4. In the embodiment of FIGS. 2A-2D, a system 50 (FIG. 2C) is comprised of a sensor chip 52 (FIGS. 2A-2B) a cassette 54, and reader 56 (FIG. 2C). A port, opening or slot on cassette 54 is configured to receive sensor chip 52. In the embodiment shown, a slot 58 is on the underside of the cassette, the slot dimensioned to receive the sensor chip so that when secured therein it is positioned for fluid communication with sample well 60. The cassette is configured for removable insertion into reader 56. In an embodiment, the cassette has electrical contacts for operable electronic contact with the contacts on the sensor chip. The cassette may optionally comprise a identifier or symbol, 62, such as a 2D bar code. The identifier 62 is positioned on the cassette so that when the cassette is inserted into the reader, the bar code can be imaged, viewed or scanned by the reader. It will be appreciated that the identifier can also be imaged, viewed or scanned by an electronic device separate from the system, and in this embodiment the identifier can be placed in positions on the cassette that do not mate or engage with the reader. The cassette can also be dimensioned for ease of handling, such as the curved region 64 dimensioned for a user's thumb or finger.

Reader 56 comprises the electronic circuitry to perform the events described herein and a power source to provide current to the sensor chip. The reader may optionally comprise one or more indicators, such a indicator light 66. The reader can optionally have a connector for engagement with an electronic device, the connector can be a male or female connector to couple the reader via a cable, USB, USB—C or other port to the electronic device. Reader 56 is shown to have a retractable USB port 68. As described below, insertion of a cassette that comprises a sensor chip into the reader initiates a series of event for performance of an assay, including signaling to a user to deposit a sample, such as sample 70 in FIG. 2D, into the sample well of the cassette.

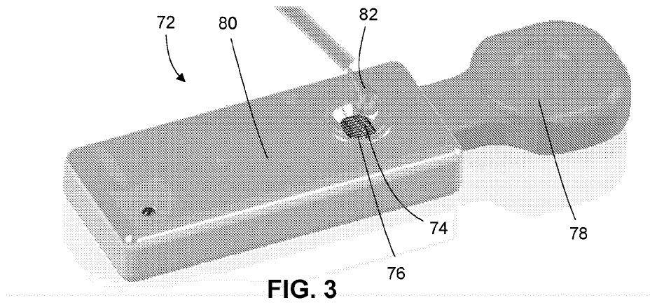

It will be appreciated that the sample port can also be placed on the reader, as shown in FIG. 3. System 72 is comprised of a sensor chip 74, visible through the sample port 76, a cassette 78, and reader 80. Insertion of cassette into the reader positions the sensor chip in fluid communication with a sample port on the reader, so that when a sample, such as sample 82, is deposited in the sample well or sample port, it contacts the sensor chip and its sensing regions.

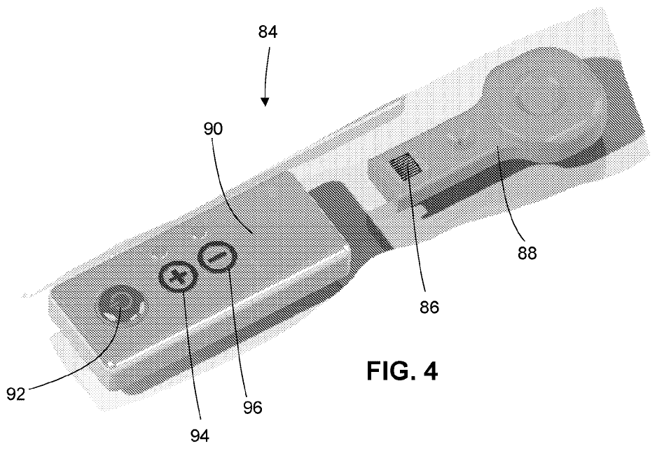

FIG. 4 shows an embodiment of a system 84 comprised of a sensor chip 86, cassette 88, and reader 90. The reader comprises a on/off switch 92 and indicators 94, 96. One of the indicators is designated as an indicator of presence of an analyte of interest, and the other is designed as an indicator of absence of an analyte of interest. In this embodiment, the reader can comprise a memory, a processor and an algorithm for analysis of resistance measurements gathered during the assay. The reader in this embodiment need not interact with or communicate with an external device.

In certain aspects, kits comprising a system described herein, optionally together with instructions for using the system, are provided. The kit can optionally include second, third and further sensor chips configures to determine presence or absence of the same or different analytes(s) of interest. The kit can optionally include a swab, sample processing reagents, and/or a container or device for sample collection.

### III. Methods of Use

Also provided are methods for detecting an analyte of interest. In an embodiment, the method comprises providing a cassette comprising a sensor chip as described herein. In an embodiment, the sensor chip is comprised of a substrate with a first planar surface and a second planar surface, electrical contacts, a sensing region comprised of a semiconducting transition metal, and a detection agent that interacts with an analyte of interest. A first resistance of the sensing region is measured, determine or evaluated, for example, by a detection or sensor in an instrument or reader that receives the sensor chip or a cassette comprising a sensor chip. A sample suspected of comprising an analyte of interest that specifically binds the detection agent on the sensor chip is deposited on the sensor chip, for contact with the sensing region. A second resistance of the sensing region is measured, determine or evaluated, for example, by a detection or sensor in an instrument or reader that receives the sensor chip or a cassette comprising a sensor chip. Based on the first and second resistance measurements, binding of the analyte of interest to the detection agent is determined. In an embodiment, if the second resistance is different from the first resistance binding of the analyte of interest to the detection agent is confirmed, and a result that the analyte of interest is present in the sample is obtained.

In another embodiment of the method a cassette comprising a sensor chip comprised of a substrate with a first planar surface and a second planar surface, electrical contacts, a sensing region comprised of a semiconducting transition metal, and a detection agent that interacts with an analyte of interest is provided. The cassette is in a reader having a port configured to receive the cassette, where the reader comprising a source of electricity, a detector, and a transmitter. A first resistance of the sensing region is measured by causing the source of electricity to apply electrical current to the electrical contacts. A sample suspected of containing the analyte of interest is deposited on the cassette to cause sample to contact the sensing region. A second resistance of the sensing region is measured by causing the source of electricity to apply electrical current to the electrical contacts. Based on the first and second resistance measurements, binding of the analyte of interest to the detection agent is determined. In an embodiment, if the second resistance is different from the first resistance binding of the analyte of interest to the detection agent is confirmed, and a result that the analyte of interest is present in the sample is obtained.

FIG. 5 sets forth an embodiment of a sequence of events involved in use of the system described herein. In the embodiment, a user has obtained or is provided with a cassette containing a sensor chip configured for detection of one or more analytes of interest. For example, a user may be concerned with a recent tick bite and desires to know whether there is an infection of Borrelia burgdorferi. Or perhaps the user is experiencing respiratory congestion and fever and desired to know if there is a respiratory infection, such as SARS-COV-2 or respiratory syncytial virus (RSV). Instructions provided to the user, either by paper in the packaging or via a bar code, QR code or the like on the packaging, guide the user to download, if needed, and/or open a software application to their mobile device. The software application guides the user though the steps of the assay protocol, including steps to collect a sample and to place it on the cassette at the right time and in the right place. Once the software application on a user mobile device is open, 100, the user is instructed to obtain a sample, if needed, although sample collection can be done at other points in the sequence of events. Then user is instructed to insert the cassette into the reader, 102. In an embodiment, insertion of a cassette into the reader activates the reader, although it is also contemplated that the reader may have an on/off switch that a user can toggle. The reader sends electrical current to the electrical contacts of the sensor chip and determines resistance of at least one, and preferably each, sensing region, 104, to determine background resistance of the sensing region(s). The user is then instructed or prompted via the software application to deposit the sample into the sample port on the cassette, 106. Sample contacts the sensing region(s) on the sensor chip, and electrical resistance of the sensing regions is measured by the reader, 108.

FIGS. 6A-6B show an embodiment of a sequence of events involved in use of the system described herein, from a user perspective. A user provided with a system or a kit comprising the system is instructed to collect a sample, which in this embodiment is done with a tool such as a swab, 110. The system or kit comprises a tube with a reagent for processing the sample, and the insert is instructed to place the tool into the tube comprising the processing reagent, 110. The tube can have a lid with a dropper tip that is placed on the tube, after the tool is removed or the stem or handle of the tool is broken to permit the lid or dropper tip to be placed on the tube, 110. The user is instructed to insert the cassette with a sensor chip into the reader, 112. The user is then instructed to dispense one or more drops from the tube of the sample onto the sample port, which can be a port on the cassette or on the reader, 114. The reader then transmits resistance measurements collected by the reader to a user's electronic device, 116; alternatively, the reader has a memory and a processor to collect and store the resistance measurement. In embodiments where the reader is configured to transmit the resistance measurements to an external electronic device, the electronic device has an algorithm to analyze the transmitted measurements and to determine whether the sample contained the analyte of interest. In embodiments, where the reader has a memory and a processor to collect and store the resistance measurement, it can also comprise an algorithm to analyze the transmitted measurements and to determine whether the sample contained the analyte of interest, and will then transmit the result of the analysis to the external electronic device. A result is displayed on the user's electronic device, 116. Then user can then remove the cassette from the reader, where the cassette can be discarded, recycled, or reused upon insertion of an unused sensor chip, 118.

As mentioned above, the sensor chip in the test cassette is configured for detection of an analyte of interest, such as an infectious agent, such as a virus, a bacterium, a fungus, or other agent, such as a therapeutic drug or a drug or substance of abuse. The analyte of interest can also be an antibody, a protein, a peptide, such as those produced in response to an infectious agent, drug, substance of abuse, or other agent. The analyte of interest can also be a nucleic acid (RNA or DNA) that indicates presence of an analyte of interest in the sample, directly or indirectly.

In embodiments, the infectious agent is a virus. Exemplary, non limiting examples, include respiratory viruses, such as influenza A (Flu A), influenza B (Flu B), respiratory syncytial virus (RSV), and a coronavirus, such as SARS-COV-2. In other embodiments, the infectious agent is a bacterial infectious agent. Exemplary bacterial infectious agents include, but are not limited to, Streptococcus pyogenes (group A Streptococcus), Staphylococcus, Neisseria meningitidis, Escherichia coli, methicillin-resistant Staphylococcus aureus (MRSA), Borrelia burgdorferi, Borrelia afzelii, and Bordatella pertussis. In certain embodiments, the infectious agent is a fungus or a mold, such as a Mucormycetes, an Aspergillus, a Pneumocystis, a Candida, or a Talaromyces.

The sample for testing can be any sample or specimen, including samples from living or deceased subjects or patients. The sample can be any biological sample, including samples from a subject or a patient or a sample from soil, water, plants, air. A “subject” or “patient” can be any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use. The soil, water, plant or air sample can be, for example, for food testing, water testing, agriculture testing or monitoring, and the like. Samples from a subject can take the form of blood, saliva, mucus, sputum, nasal, urine, sweat, fecal, and the like.

In embodiments, the system achieves a sensitivity and/or specificity for detection of an analyte of interest that is comparable to that provided polymerase chain reaction (“PCR”) analysis of the same sample for the same nucleic acid analyte of interest. For example, in an embodiment, the system is capable of detecting a nucleic acid marker for, for example, SARS-COV-2 or influenza A, on the order of 0.01 to 10 pg/mL in the provided sample. It will be appreciated that the system is capable of detection of the analyte of interest above this range of 0.01-10 pg/mL, as there is no upper limit on amount or quantity of the analyte of interest that can be present in the sample. For example, a system capable of detecting presence or absence of human chorionic gonadotropin is contemplated, where the level of this protein a sample can be in the milliIU/L range.

In another aspect, a computer-implemented method for determining presence or absence of an analyte of interest in a sample is provided. The method comprises obtaining a signal from an electrical activity associated with a sensor chip; analyzing, using a processor, the signal to determine a change in electrical activity relative to a baseline signal of electrical activity in the absence of a sample; determining, using the processor, presence or absence of an analyte of interest, and transmitting a test result indicating presence or absence of the analyte of interest, or of a condition, disease or disorder based on presence or absence of the analyte of interest, to a user or other party.

In another aspect, a method to determine presence or absence of one or more analyte(s) of interest in a sample, or to diagnose a condition, disease or disorder based on presence or absence of one or more analyte(s) of interest in a sample, are provided. The method comprises providing to a subject a system comprised of (i) a sensor chip, optionally with a cassette for handling, the sensor chip comprising one or more electrical contacts and one or more sensing regions, each sensing region comprising a detection reagent for the one or more analyte(s) of interest; and (ii) a reader configured to receive the sensor chip, or its optional cassette, and comprising circuitry that operably engages the one or more electrical contacts on the sensor chip, a source of electrical current, and a sensor to detect resistance in the one or more sensing regions, where the circuitry is configured to process an electrical signal from the one or more sensing regions, and (iii) a software application comprising an algorithm for evaluating the signal, and instructing the subject to (i) install the software application on a computing device, (ii) insert the sensor chip into the reader and (iii) to place a sample onto the sensor chip, whereupon instructions stored on the reader or in the software application on the computing device, initiate an assessment of resistance, before sample placement and after sample placement. Electrical activity associated with resistance is determined and transmitted to a processor on the computing device for processing by an algorithm in the software application, to determine presence or absence of the one or more analytes of interest.

A simplified user process flow comprises, for example, the following steps: (1) collecting a sample, optionally with a sample collection tool, such as a finger prick device to obtain blood, a pipette to obtain a fluid, a spatula to obtain a soil sample, or a swab to obtain a nasal or throat sample; and optionally treating the sample with a reagent to prepare it for analysis, such as by inserting a nasal swab specimen into a tube with extraction reagent and mixing well, (afterwards, the swab may be discarded); (2) inserting a cassette comprising a sensor chip into a reader; (3) dispensing an aliquot, such as a drop, of sample or of the processed or treated sample in a solution onto or into a sample port on the cassette. As a result of these steps by a user of the system, a test result is then transmitted to a user device, e.g., a smartphone, via a software application on the device. After that, the test cassette can be removed from the reader and properly discarded if the cassette is a single use cassette. If it is not, the cassette can be retained and a fresh sensor chip inserted into the cassette.

In some embodiments, when the cassette is inserted into the reader, the reader scans an identifier or symbol, such as a QR code or bar code or indicia on the sensor chip or the cassette, to confirm the test or assay type, to call up a test method file (with analysis algorithms) for the test type, to optionally confirm the expiration date of the test, to collect/transmit identifier data in the identifier or symbol (if collected), and to transmit resistance/result data to an end user, such as a user's mobile device or computing device. The software application with its analysis algorithm analyzes the data based on the assay type and reports a result for each assay and/or for each analyte.

### D. Methods of Manufacture

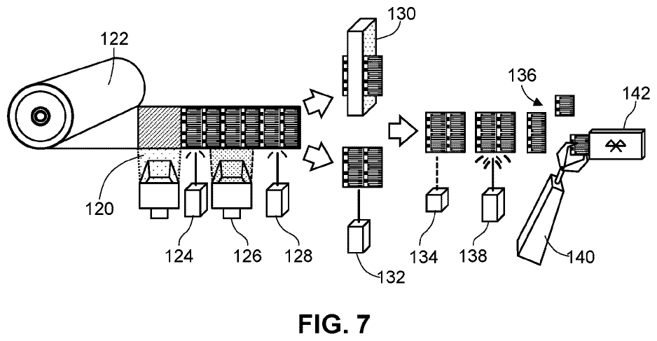

FIG. 7 illustrates a high-throughput process for production of sensor chips described herein. In the process, a layer or film of a conductive metal (e.g., Cr, Ti, Scn Ni, V, Hf, W, Nb, Au, Ag, Cu and Pt, or combinations thereof) is deposited, 120, onto a substrate, shown in this embodiment as a flexible glass sheet, 122. The layer of conductive metal is patterned, such as by illumination or laser etching, 124, to remove regions of the conductive metal layer to thereby form an array of electrical leads in a desired pattern, e.g., with one or more gaps in one or more leads, with any number of leads in the array, with any desired number of leads that will be ground leads vs. leads with a sensing region. Transition metal, or a compound comprising a transition metal, such as MoS₂, is deposited over the array of electrical leads, 126. A laser is used to form a sensing region in a gap of an electrical lead, 128, to cause the transition metal, or a compound comprising a transition metal, to become semiconductive or conductive. For example, MoS₂ exposed to a laser becomes crystalline and semiconductive. Laser patterning of the transition metal, or a compound comprising a transition metal, is done in a manner to leave intact the metal material for connecting electrical leads, 130 and 132, and to render the transition metal, or a compound comprising a transition metal, in the sensing region to become semiconducting or conducting. An alternative to laser exposure is thermal treatment to anneal the transition metal, or a compound comprising a transition metal, and render it semiconducting or conducting. A detection reagent is then deposited in each sensing region, 134, via an automated dispensing system. An exemplary detection reagent is an rFab with a linker, such as an rFab-HLL fusion protein, where the rFab has selective and/or specific binding for an antigen or analyte of interest in a sample. Individual sensor chips, 136, are cut, 138, from the substrate, for example with a laser. Optionally, a pick-and-place robot, 140, is used to insert each individual sensor chip into a cassette 142 that can be packaged for shipping.

In an embodiment, the electrical leads and sensing region on a sensor chip are fabricated by depositing, or sputtering, in a uniform layer of a conductive metal across a first surface of a substrate. In an embodiment, the uniform layer has a thickness of about 50 nm, or between about 30-80 nm, or between about 25-100 nm. In an embodiment, the conductive metal is gold. In an embodiment, the substrate is a flexible glass sheet, which may have dimensions of 300 mm×300 mm. In an embodiment, the sputtering is done under vacuum. Next, a scribing laser ablates the conductive metal to form a pattern of leads and contacts, the pattern can be specified in an electronic drawing file transferred to the laser system. This forms electronic leads and contacts as part of the sensor chip. Then, a transition metal or a compound comprising a transition metal, such as, respectively, pure molybdenum or molybdenum disulfide, is deposited, generally such that 3-8 atomic layers of are deposited. A scribing laser removes the transition metal or a compound comprising a transition metal that is not needed, ensuring that the material that resides in a 0.1 mm gap within each lead is retained. This retained material is treated, with a laser or with heat, to render it semiconducting. For example, with the retained material is MoS₂, the sensor chip is heated by a laser and/or by exposure to heat to anneal or crystalize the MoS₂, transforming the amorphous MoS₂ into a crystalline two-dimensional transducer. In an embodiment, the sensor chip is treated by exposure to a laser and to heat, such as in a furnace, to yield sensor chips comprised of, in an embodiment, up to 9-independent sensors per chip.

The sensor chips are then processed to deposit in each sensing region a desired reagent. In an embodiment, a nanoliter or picoliter automated spotter instrument is used to deposit a detection agent (also called a reagent), or a mixture of assay specific reagents. An example is monoclonal antibody (“mAb”) expressed recombinantly in the form of a recombinantly expressed FAB (“IFAB”) that contains at least one linker, such as an amino acid peptide (e.g., “HLL”) that is capable of binding to the surface of the sensor. In an embodiment, a linker is attached to each heavy chain of the mAb or rFAB. After depositing the reagent, the sensing chips are laser singulated from the array formed on the substrate in an automated system and stored until needed.

In certain embodiments, the reagent deposited in each sensing region may further comprise other reagents, including, but not limited to, salts, sugars, and other agents to enable long-term room temperature stability with rapid rehydration. In embodiments, the method of manufacture comprises sputtering pure molybdenum (Mo) or MoS₂ metal in a uniform 50 nm layer across the surface of a flexible glass sheet under high vacuum, and ablating the molybdenum or MoS₂ metal except the pattern of leads and contacts specified in an electronic drawing file transferred to the laser system using a scribing laser. As a result, the glass sheet may be coated with 3-8 atomic layers of semiconducting MoS₂ and laser-patterned thereby forming leads and contacts as part of the sensor chip. In certain embodiments, the scribing laser removes the MoS₂ that is not needed, retaining only the MoS₂ at the sensor location which resides in gap within each lead. In embodiments, the gap is about 0.1 mm or is between about 0.02-0.2 mm, 0.04-0.18 mm, or 0.05-0.16 mm, or 0.08-0.14 mm. Methods for manufacture are described, for example, in U.S. Patent Application Publication Nos. 2021/0299781; 2021/0301381; 2021/0299789; 2021/0301388; 2022/0062984; and 2023/0045818, each incorporated by reference herein.

In some embodiments, the method may further comprise heating the sensor chip with a laser to anneal or crystalize the MoS₂, thereby transforming the amorphous MoS₂ into a crystalline two-dimensional transducer; and functionalizing the sensors by spotting a mixture of assay specific reagents comprising rFABs, thereby spotting the substrate, such as a glass sheet, with a detection agent, such as an antigen binder that is specific for an analyte of interest, such as infectious agents.

The sensor functions when the sample is applied to the sensor chip that contains the binding antibody. The target antigen (also referred to as an analyte of interest), if present, binds to the rFAB which brings the antigen close to the sensor surface. The antigen carries a “natural” charge that is detected by the sensor as a change in the resistance of the sensor that is detected by a resistance detector to which the sensor is attached. In an embodiment, the sample to answer time is less than 5 minutes, less than 4 minutes, less than 3 minutes or less than 2 minutes. No change in resistance would be observed with a negative sample that does not contain the target analyte (or analyte of interest).

In an embodiment, the substate of the sensor is a flexible glass sheet with a flexible polymer backing. In an embodiment, the polymer backing is adhered to the substrate with an adhesive. In an embodiment, the substrate is a flexible glass, with or without a flexible polymer backing, with at least one surface treated prior to deposition of materials to form the sensor chip thereon with a laser to polish and/or reduce roughness of the substrate surface. In an embodiment, the substrate surface on which metals, transition metals, detection agents are to be deposited is treated by exposing the surface to a focused laser beam at an energy to cause a layer of the substrate surface to melt. The melted layer will hardens to yield a surface that has improved (e.g., reduced) surface roughness that the surface roughness prior to exposure to the focused laser. In an embodiment, the substrate surface on which metals, transition metals, detection agents are to be deposited is first treated by exposing the surface to an out of focus laser beam. Subsequent to the exposure to the out of focus laser beam, the surface is further exposed to a focused laser beam at an energy to cause a layer of the substrate surface to melt. The melted layer will hardens to yield a surface that has improved (e.g., reduced) surface roughness that the surface roughness prior to exposure to the out of focus and focused laser. In embodiments, the surface roughness after treatment is as described herein above.

In an embodiment, an array of sensor chips is fabricated at a scale of 10,000 to 60,000 sensor chips per lot and/or on a roll of a substrate, where the roll can be any size between approximately 2 m×20 m and 300 mm×20 meters, yielding >66,000 sensor chips per roll. As an example, an array of sensor chips can be fabricated using a 2 meter by 20 meter roll of substrate, where each sensor chip has 5 to 9 sensors or sensing regions per sensor chip, to yield 750,000 to 1,000,000 sensor chips produced in a single manufacturing run. During fabrication, the array of sensor chips can be inspected for quality control using, for example, Raman spectroscopy, using back scatter laser light after annealing of sensors and with software developed to analyze and grade each scan. The sensor chips are activated or functionalized with a detection agent using a robotic automated pipetting system and/or automated ink jet system designed for protein containing solutions, including a nanoliter and/or a picoliter sized spotting drops and using one or many small volume drops. In an embodiment, an indicator, such as a dye (colored, fluorescent, luminescent, etc) can be added to the solution comprising the detection agent. A high speed optical device using an AI program can identify out-of-specification senor chips. Sensor chips in the array are singulated from the master roll into individual sensor chips to enable pick and place into test cassettes using a singulation laser system. A quality control cassette can be used to confirm leads and contacts are showing conductivity for a certain percentage of each finished sensor chip cassette lot.

In embodiments, the sensors, such as biosensors, provided herein comprise MoS₂ that can be formed into two dimensional sheet of a semiconducting sensor (meaning length and width but effectively no depth, e.g., only about 3-8 atoms/atomic layers thick). In some embodiments, the sensors provided herein comprise other 2D materials, including but not limited to graphene. In some embodiments, MoS2 provides superior sensor performance when compared to graphene and other 2D materials.

In embodiments, the sensor is operated by applying a direct current voltage across the sensor followed by depositing a sample or specimen onto the sensor (or into a well of a cassette containing one or more sensors). In embodiments, the sensors are made using a process similar to that of producing microchips. For example, in some embodiments evaporated metal (sputtering) is uniformly coated onto sheets of thin flexible glass. Such processes enable production of large volumes of sensor chips at low cost.

In embodiments, an industrial laser is used to ablate or reevaporate the sputtered metal and leave behind only the material needed for the sensor. For example, the metal that is left behind is in the form of leads comprising a gap between ends of the leads.

In embodiments, an additional layer of material is applied to the previously coated and ablated sensor, including application of the additional material across the gap between ends of the leads. In embodiments, the additional material is a transition metal or a compound comprising a transition metal, such as MoS₂, that is applied as an insulator and can be activated and/or transformed into a semiconductor by exposure to a heat and/or a laser. In embodiments, the activated semiconductor material, such as laser treated MoS₂ is present in the gap between the leads formed by the sputtered and ablated conductive metal material.

In embodiments, the gap between conductive leads comprising a semiconductor material further comprises a detection agent (also called a biomolecule) on the surface of the semiconductor for analysis of an applied sample for the presence of an analyte of interest. In embodiments, an analyte of interest interacts with the detection agent present in the gap to cause a change in the conductivity or resistance across the sensor gap in a measurable manner. For example, the sensor may comprise an antibody, peptide, sequence of nucleotides, or other detection agent on the surface of the sensor in the sensing region within the gap between the leads. Binding of a target analyte of interest present in an applied sample causes a change in the conductivity or resistance across the gap, the change being relative to the conductivity or resistance across the gap before the sample is applied to the sensor chip or before the sample contacts the gap (e.g, the sensing region that comprise the detection agent). Accordingly, a detected change in conductivity or resistance across the gap indicates the presence of the analyte of interest in the applied sample.

In embodiments, a thin flexible substrate, such as flexible glass, enables traditional foundry equipment to be used to support a high volume reel to reel biosensor manufacturing processes. In embodiments, roll and unroll techniques may also be used to deposit a detection agent onto the sensor chips to ‘activate’ or ‘functionalize’ the sensors. In some embodiments, the detection agent(s) is/are deposited using a spotting instrument, to deposit a drop or drops comprising a detection agent, onto the sensing region. The method of manufacture provides advantages including storage of sensors for long periods, such as storage in roll form. In some embodiments, sensors, including rolls of sensors, are also amenable to transport to other locations for subsequent and/or additional production steps. For example, glass, such as thin flexible glass, may be coated in one location, ablated in another, and activated and/or finally assembled, including addition of a detection agent and assembly into a cassette or pill-like format, in yet another location.

In embodiments, during the fabrication process the substrate, such as a flexible glass sheet, is initially unrolled and coated with a 1 to 1,000 atom thick layer of sputtered material (for example a 5 atom thick layer of gold). In some embodiments, the sheet is rerolled and a laser(s) removes (ablates) unwanted conductive material (such as removal of gold or another conductive metal compatible with this layer of the sensor) to form conductive leads in predefined dimensions/locations. In embodiments, the coated/ablated sheet is unrolled the exposed/ablated glass surface is then coated with a 1 to 1,000 atom think layer of an insulator, such as a 5, 6, 7, or 8, or 5-8, atom thick layer of insulator, such as MoS₂, which is applied in the form of an insulator (i.e., a material that inhibits current flow). In some embodiments, the sheet is then rerolled and a laser anneals (i.e., heats the insulator material to form a semiconductor from the insulator) the insulator, such as MoS₂, at each sensor site to form individual sensors of organized insulator, such as organized MoS₂, at the junction between the ground and current leads, wherein the insulator is transformed to a semiconductor by the laser between the ground and current leads.

In some embodiments, the sensor chips analyze a sample for the presence or absence of an analyte of interest without the need for the sample to flow from one portion of the sensor to another. That is, the sample that remains stagnant or stationary during the analysis of the sensing region, where the analysis can include incubation of the sample for a period on the surface of the sensor chip. Accordingly, the sensor chips do not rely on sample flow and/or any challenges or constraints associated therewith. Accordingly, systems using the biosensors described herein represent an improvement over standard microfluidic or lateral flow technologies at least because they do not require movement of the fluid sample from one zone to another.

In some embodiments, the biosensors described herein include binding agents, such as antigen(s) and/or antibodies, including capture antibodies, bound to a sensor surface in an immobilized fashion. In some embodiments, when an analyzed sample comprises the target analyte, the target analyte binds the immobilized binding reagent, such as an antigen or antibody, that is bound to the sensor surface. Binding of a target analyte to a binding agent immobilized on the sensor surface generates a change in resistance on the sensor surface that is detected, for example, by a current or resistance detection device.

In some embodiments, the change in resistance on the sensor surface is transmitted from the reader to an associated software application (also referred to herein as an App). In some embodiments, Apps that may be used in conjunction with reading a change in resistance on the surface of a sensor chip are designed and/or configured to be used on electronic device, such as a mobile device and/or personal computer, to collect resistance data and/or to receive resistance data. In some embodiments, collected or received resistance data is analyzed by an algorithm in the App. In some embodiments, a final result indicating the presence of absence of an analyte of interest, or a final result indicating a positive or negative for a condition, disorder or disease, in the analyzed sample is provided by the App on the electronic device subsequent to analysis of the data related to change in resistance on the sensor surface.

In some embodiments, a sensor chip can perform multiple analyses, such as measuring resistance changes (e.g., measuring resistance across the sensor every 5 to 10 seconds starting when the sample is added and continuing as long as needed, such as for about 90 seconds to about 5 minutes) depending on the assay. For example, in some embodiments, resistance may be measured every about 5 seconds, about 6 seconds, about 7 seconds, about 8 seconds, about 9 seconds, about 10 seconds, or more or less often as needed for appropriate for analysis of the particular analyte. Such repeated measurements may, in some embodiments, be performed for about 90 seconds, about 2 minutes, about 3 minutes, about 4 minutes, about 5 minutes, or for longer or shorter durations as appropriate for analysis of the particular analyte. In some embodiments, data related to the change in resistance is analyzed by an algorithm, such as an algorithm designed for analysis of the specific target analyte, to present a final test result to the user.

In some embodiments, the sensor chip can provide direct detection of binding between a detection agent immobilized on the sensor chip and an analyte of interest in the sample without the need for additional steps or reagents. For example, the sensor chip yields a detection analysis without a second, or additional, detection step as is required in a standard ELISA or in the use of colored or fluorescent detection beads in a lateral flow assay. The sensor chip can be used at room temperature and can accommodate a wide ranges of specimen volumes and types. Binding between the detection agents and the analyte(s) of interest is directly detected without additional manipulation or addition of other reagents.

In some embodiments, the biosensors provided herein comprise an electronic sensor chip that employs a 1 to 1,000 atom layers thick, such as about 1 atom, about 2 atoms, about 3 atoms, about 4 atoms, about 5 atoms, about 6 atoms, about 7 atoms, about 8 atoms, about 9 atoms, about 10 atoms, about 20 atoms, about 30 atoms, about 40 atoms, about 50 atoms, about 60 atoms, about 70 atoms, about 80 atoms, about 90 atoms, about 100 atoms, about 200 atoms, about 300 atoms, about 400 atoms, about 500 atoms, about 600 atoms, about 700 atoms, about 800 atoms, about 900 atoms or about 1,000 atoms thick, 2-dimentional MoS₂ sensor chip or biosensor. In some embodiments, the sensor chips herein comprise a sensing region with a 3-5 atom thick 2-dimentional MoS₂ for femtogram sensitivity.

As can be appreciated, the sensor chip provides, in embodiments, femtogram/mL sensitivity in a less than 5 minute, 4 minute, 3 minute, 2 minute or 1.5 minute time periods. The sensor directly detects the binding of an analyte of interest (e.g., an antigen, antibody, nucleic acid, etc.,) in a sample to a detection agent immobilized on the sensor chip without the need for a separate detection step. The direct binding of the binding of an analyte of interest to a detection agent immobilized on an individual sensing regions (or sensors) is by a change in conductivity, current or resistance. In embodiments, the sensing region is positioned between two electrical leads, a positive and negative, in a configuration to complete a circuit. This circuit enables measuring the change in resistance during the assay as the analyte of interest binds to the detection agent. The binding event brings the analyte of interest close to the sensor surface resulting in changes in the resistance. The change in resistance can be a reduction or an increase relative to the resistance absent binding by an analyte of interest (i.e., background resistance). The sensor can have any configuration or layout, depending on the number of analytes to be detected in a sample. By way of example, a sensor chip with 8-12 sensing regions for detection of 7-11 analytes of interest plus a negative control can be used to determine if a sample contains certain infectious agents, for example, in determining cause of a respiratory infection.

An assay using a sensor chip is performed by adding a liquid specimen to the surface of the sensor chip in a way that the liquid sample covers all sensing regions on the chip. Then without mixing or any other manipulation and no other reagents, each sensor that binds an analyte of interest will register a change in resistance without interference or cross talk with other sensors.

### IV. EXAMPLES

### Example 1

Exemplary System with a Sensor Chip

An electronic sensor chip to measure changes in the electrical conductivity or resistance of a layer (e.g., 3-5 molecular layer) transitional metal, such as molybdenum or MoS₂, caused by binding or interaction of an analyte of interest in a sample to or with a detection agent attached to or associated with the thin layer of transitional metal is provided. The sensor chip offers a reportable test result in less than about 6 minutes or less than about 5 minutes or less than 4 about minutes or less than about 3 minutes or less than about 2 minutes or less than 1 minute, the time being measured from when a sample is placed on the sensor to when the test result is available to a reader for the sensor.

The system also comprises a portable, battery-operated reader with battery replacement or external recharging capabilities and a downloadable software application. The system is designed for home, doctor's office, hospital, laboratory, clinic, and related uses to allow for result sharing with physician/medical team, governmental agencies, clinical sites, where needed, the sharing is deidentified data.

The 2D MoS₂ sensor chip, in an embodiment, meets criteria needed to significantly improve rapid testing for respiratory viruses, such as but not limited to, Flu A, Flu B, RSV, and COVID. With improved analytical sensitivity between 0.01 and 10 pg/mL for protein targets like Flu A nucleoprotein, shortened process times (2-4 minutes) are required for extracting the sample, running the assay, and receiving the result. A two-log improvement in analytical sensitivity is provided, in an embodiment, thus approaching PCR performance.

Table 1 compares a nitrocellulose based immunoassay to a MoS₂ sensor chip.

TABLE 1
Nitrocellulose ImmunoAssays vs. MoS₂ Assays

| | Nitrocellulose | |
|---|---|---|
| | Immunoassay | MoS₂ Sensor Chip |
| Time to Test | 5-20 minutes | ≤2 minutes |
| Ease of Use | Simple | Simple |
| Stability | Store at room | Store at room |
| | temperature for | temperature for |
| | 12-24 months | 12-24 months |
| Target Analytes | 1-4 | 1-12 |
| Limit of Detection | Visual >1 ng/mL | 0.01 pg/mL |
| | Fluorescent >50 pg/mL | |
| Sensitivity/ | 85%-95% | ≥95% |
| Specificity | | |
| Results | Qualitative for visual | Objective |
| | assays (Subjective) | |
| Technology | Sandwich Immunoassay | Direct Binding Assay |
| Antibody Binding | Non-Specific Orientation | Specific Orientation |

MoS₂ has a sensitivity advantage over other a nitrocellulose based immunoassay technologies. Data from studies have shown that the 0.25 mm×0.25 mm sensor can detect a change above background when a viral protein binds to an rFAB-HLL capture reagent of about 250 individual proteins or about 10 fgm/mL.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A system, comprising:
(a) a cassette comprising a sensor chip, the sensor chip comprised of a substrate with a first planar surface and a second planar surface, electrical contacts, a sensing region comprised of semiconducting transition metal, and a detection agent that interacts with an analyte of interest; and
(b) a reader with a port configured to receive the cassette, the reader comprising a source of electrical current, a detector and a transmitter.

2. The system of claim 1, wherein the detector is capable of converting resistance of the sensing region to electronic signal.

3. The system of claim 1, wherein the detector is capable of detecting resistance of a sensing region on the sensor chip.

4. The system of claim 1, wherein the transmitter is capable of transmitting an electronic signal.

5. The system of claim 1, wherein the transmitter is a transceiver.

6. The system of claim 1, wherein the transmitter or transceiver is wireless.

7. The system of claim 1, wherein the transmitter or transceiver comprises a wired or physical connection.

8. The system of claim 1, wherein the source of electrical current is capable of applying direct current or alternating current.

9. The system of claim 1, wherein the source of electrical current is capable of applying from about 1 pA to 100 nA to the electrical contacts.

10. The system of claim 1, wherein the reader is configured for insertion into a universal serial bus (USB).

11. The system of claim 1, wherein the reader is configured to power on in response to insertion of a cassette into the port.

12. The system of claim 1, wherein the reader comprises a memory and a processor.

13. The system of claim 12, where the reader comprises an algorithm stored in memory, the algorithm being initiated in response to (i) insertion of a cassette into the port or (ii) placement of a sample onto the cassette.

14. The system of claim 13, wherein the algorithm instructs the transmitter to transmit a signal to an electronic device that is separate from the system.

15. The system of claim 14, wherein the electronic device is a user mobile device.

16. The system of claim 1, wherein the system further comprises a software application downloadable to an electronic device separate from the system, the software application configured to respond to signal transmitted from the reader.

17. The system of claim 16, wherein the electronic device separate from the system comprises a memory and a processor, wherein the software application is retained in the memory and comprises an executable file comprising an algorithm with instructions for interaction between the reader and the external device separate from the system.

18. The system of claim 1, wherein the substrate is a flexible glass.

19. The system of claim 1, wherein the electrical contacts are comprised of a conductive metal.

20. The system of claim 19, wherein the metal is selected from the group consisting of Cr, Ti, Sn Ni, V, Hf, W, Mo, MoS₂, Nb, Au, Ag, Cu and Pt, and combinations thereof.

21. The system of claim 1, wherein the transition metal is selected from the group consisting of molybdenum, tungsten, niobium, tantalum, vanadium, titanium, chromium, iron, rhodium, hafnium, rhenium and mixtures thereof.

22. The system of claim 1, wherein the transition metal further comprises an element selected from the group consisting of nitrogen, sulfur, selenium, phosphorous, hydrogen and carbon.

23. The system of claim 1, wherein the transition metal is molybdenum.

24. The system of claim 1, wherein the sensing region is comprised of molybdenum disulfide (MoS₂).

25. The system of claim 24, wherein the molybdenum disulfide is crystalline.

26. The system of claim 1, wherein detection agent has specific binding for the analyte of interest.

27. The system of claim 1, wherein the detection agent is an antibody fragment.

28. The system of claim 1, wherein the detection agent is a fusion protein of a linker and an antibody fragment.

29. The system of claim 1, wherein the detection agent is a fusion protein of two linkers and an antibody fragment.

30. The system of claim 29, wherein the antibody fragment comprises a first heavy chain and a second heavy chain, wherein one of said two linkers is attached to a terminal end of the first heavy chain and one of said two linkers is attached to a terminal end of the second heavy chain.

31. The system of claim 29, wherein the antibody fragment comprises a constant heavy chain and a constant light chain, and one of said two linkers is attached to the constant heavy chain and one of said two linkers is attached to the constant light chain.

32. The system of claim 1, wherein the cassette and/or the sensor chip comprises an identifier that encodes information regarding the sensor chip.

33. The system of claim 32, wherein the information identifies an analyte of interest with which the detection agent interacts.

34. The system of claim 1, wherein the cassette comprises contacts for operable connection with the source of electrical current in the reader.

35. The system of claim 1, wherein the cassette comprises a plurality of sensor chips.

36. The system of claim 1, wherein the sensor chip comprises a plurality of sensing regions.

37. The system of claim 1, wherein the sensor chip comprises a plurality of sensing regions, wherein each sensing region comprises an immobilized detection agent.

38. The system of claim 36, wherein the detection agent in each sensing region of the plurality of sensing regions interacts with the same analyte of interest.

39. The system of claim 36, wherein the detection agent in each sensing region of the plurality of sensing regions interacts with a different analyte of interest.

40. The system of claim 36, wherein the plurality of sensing regions comprises between 3-25 sensing regions.

41. The system of claim 36, wherein each sensing region in the plurality of sensing regions has a dedicated pair of electrical contacts.

42. The system of claim 1, wherein the sensor chip comprises a plurality of sensing regions, wherein at least one sensing region comprises a detection agent that serves as an assay control.

43. The system of claim 1, wherein the cassette comprises contacts for operable connection with the source of electrical current in the reader.

44. The system of claim 1, wherein the reader further comprises one or more indicators.

45. The system of claim 44, wherein the one or more indicators is a light, an audible signal and/or a sensory signal.

46. The system of claim 1, wherein the cassette comprising a component to provide heat to the sensor chip.

47. A method for detecting an analyte of interest, comprising:
providing a cassette comprising a sensor chip comprised of a substrate with a first planar surface and a second planar surface, electrical contacts, a sensing region comprised of a semiconducting transition metal, and a detection agent that interacts with an analyte of interest;
evaluating a first resistance of the sensing region; and
contacting the sensing region with a sample suspected of comprising an analyte of interest that specifically binds the detection agent;
evaluating a second resistance of the sensing region; and
detecting binding of the analyte of interest to the detection agent if the second resistance is different from the first resistance.

48. A method for detecting an analyte of interest, comprising:
providing a cassette comprising a sensor chip comprised of a substrate with a first planar surface and a second planar surface, electrical contacts, a sensing region comprised of a semiconducting transition metal, and a detection agent that interacts with an analyte of interest;
inserting the cassette in a reader having a port configured to receive the cassette, the reader comprising a source of electricity, a detector, and a transmitter;
measuring with the detector a first resistance of the sensing region by causing the source of electricity to apply electrical current to the electrical contacts;
depositing a sample, or instructing to deposit a sample, on the cassette to cause sample to contact the sensing region;
measuring with the detector a second resistance of the sensing region by causing the source of electricity to apply electrical current to the electrical contacts; and
detecting binding of the analyte of interest to the detection agent if the second resistance is different from the first resistance.

49. The method of claim 47, further comprising transmitting with the transmitter data associated with the first resistance, the second resistance, or both to an electronic device.

50. The method of claim 49, wherein the electronic device comprises a memory and a processor, and retained in the memory is a software application comprising an algorithm for analyzing the data.

51. The method of claim 47, wherein binding of the analyte of interest to the detection agent is detected if the second resistance is lower than the first resistance.

52. The method of claim 47, wherein binding of the analyte of interest to the detection agent is detected if the second resistance is higher than the first resistance.

53. The method of claim 47, wherein the depositing a sample, or instructing to deposit a sample, comprises depositing a sample selected from the group of blood, urine, sweat, mucus, nasal discharge, saliva, feces, soil, waste stream, and food.

54. The method of claim 47, wherein the depositing a sample, or instructing to deposit a sample, comprises depositing a sample selected from the group of blood, urine, sweat, mucus, nasal discharge, saliva, and feces that has been processed prior to said depositing.
